(19) United States
(12) Patent Application Publication
(10) Pub. No.: US 2016/0030890 A1
(43) Pub. Date: Feb. 4, 2016

(54) CONTAINER WITH BIOFILM FORMATION-INHIBITING MICROORGANISMS IMMOBILIZED THEREIN AND MEMBRANE WATER TREATMENT APPARATUS USING THE SAME

(71) Applicant: SNU R&DB FOUNDATION, Seoul (KR)

(72) Inventors: Chung-Hak Lee, Seoul (KR); Seon-Ki Lee, Seoul (KR); Kibaek Lee, Chungcheongnam-do (KR); Sang Hyun Lee, Gyeonggi-do (KR); Dong-Chan Choi, Seoul (KR)

(21) Appl. No.: 14/883,354

(22) Filed: Oct. 14, 2015

Related U.S. Application Data
(63) Continuation-in-part of application No. 13/879,495, filed on Apr. 15, 2013.

(30) Foreign Application Priority Data
Oct. 15, 2010 (KR) 1020100101114
Sep. 29, 2011 (KR) 1020110099110
Sep. 16, 2015 (KR) 1020150130886

(51) Int. Cl. B01D 65/08; C02F 1/44; C02F 3/34; C12N 11/04; C02F 3/12

(52) U.S. Cl. CPC B01D 65/08; C12N 11/04; C02F 3/1273; C02F 3/342; C02F 3/348; C02F 1/444; B01D 2321/166

(57) ABSTRACT

The present disclosure relates to a technique for inhibiting biofouling of the surface of a membrane caused by a biofilm, through immobilizing biofilm formation-inhibiting microorganisms to a container in a membrane water treatment process. The present disclosure provides a non-hollow/hollow columnar or sheet-like permeable carrier with flowability owing to submerged aeration and a container with biofilm formation-inhibiting microorganisms immobilized therein, comprising biofilm formation-inhibiting microorganisms immobilized in the carrier. The present disclosure also provides a membrane water treatment apparatus comprising a reactor accommodating water to be treated, a membrane module for water treatment and a container with biofilm formation-inhibiting microorganisms immobilized therein placed in the reactor.

### RELATED APPLICATIONS

This application is a continuation-in-part of and claims the benefit of priority under 35 U.S.C. §120 to U.S. patent application Ser. No. 13/879,495, filed on Apr. 15, 2013, which is a nationalization under 35 U.S.C. §371 from International Application No. PCT/KR2011/007666, filed Oct. 14, 2011 and published as WO 2012/050392 A2 on Apr. 19, 2012, which claims the priority benefit of Korean Application No. 10-2010-0101114, filed Oct. 15, 2010; and Korean Application No. 10-2011-0099110, filed Sep. 29, 2011, the contents of which applications and publication are incorporated herein by reference in their entirety. This application also claims the priority benefit of Korean Application No. 10-2015-0130886, filed Sep. 16, 2015, the contents of which is incorporated herein by reference in its entirety.

### TECHNICAL FIELD

The present disclosure relates to a technique for inhibiting membrane biofouling caused by a biofilm formed on the membrane surface during a membrane water treatment process. More particularly, the present disclosure relates to a container in which microorganisms capable of inhibiting biofilm formation are immobilized and a membrane water treatment apparatus including the same inside a reactor for water treatment, so as to maintain stably the permeability of the membrane for a long period of time.

### BACKGROUND ART

Recently, a membrane process has been applied in various water treatment processes to obtain high-quality purified water. In addition to the membrane bioreactor (MBR) process which combines a membrane separation process with a biological water treatment reactor, the conventional membrane water treatment process combined with a physical/chemical pretreatment process, and nanofiltration and reverse osmosis membrane processes for advanced water treatment have been actively researched and widely applied in actual processes.

During the operation of the membrane process, microorganisms such as bacteria, molds and algae that exist in the reactor start to attach and grow on the membrane surface (attached growth) and finally form a film with a thickness of around a few tens of micrometers, i.e. a biofilm, that covers the membrane surface. The biofilm formation is frequently observed not only in the membrane bioreactor process but also in the conventional membrane water treatment process and the advanced water treatment processes such as nanofiltration and reverse osmosis membrane processes. This biofilm causes membrane biofouling, which serves as filtration resistance to degrade the filtration performance of the membrane and thus leads to problems of decreased permeability, such as shortening of the cleaning cycle and lifespan of the membrane and increase of energy consumption required in filtration and, ultimately, deterioration of the economic efficiency of the membrane water treatment process. Not only in the membrane water treatment process, biofilm or slime is also formed on a material surface by microorganisms existing in water systems such as water tanks or water pipes of buildings and industrial facilities, thereby degrading performance of equipment (e.g., corrosion of metal surfaces, degradation of cooling tower efficiency and contamination of pipe networks by microorganisms) or deteriorating external appearance.

Various researches have been done in the past 20 years to solve the above-described problems. However, the biofilm formed naturally by microorganisms on a surface in contact with water is not completely removed by the conventional physical (e.g., aeration) or chemical methods (e.g., injection of chemicals such as a chlorine compound) and a satisfactory solution for prevention/control of membrane biofouling using conventional physical/chemical methods has not been suggested yet. The outstanding membrane biofouling problem is attributed to the lack of understanding and technical consideration of the characteristics of microorganisms in the reactor that directly and indirectly affect the membrane biofouling in membrane water treatment process.

The biofilm which is a major cause of membrane biofouling in the membrane water treatment process is not easy to remove once it is formed, because it has high resistance to external physical and chemical impacts. As a result, although several conventional techniques for inhibiting membrane biofouling by physical and chemical methods are effective in the initial stage of biofilm formation, the effect of inhibiting biofouling decreases after maturation of the biofilm. In order to overcome the problem of the conventional methods, development of a new technology approachable from the viewpoint of characteristics of the microorganisms in the reactor, especially regulating and controlling the formation and growth of biofilm on the membrane surface, is required. However, there have been no fundamental solutions based on research on the characteristics of microorganisms in addition to the physical/chemical methods.

Meanwhile, microorganisms tend to respond to environmental change such as temperature, pH, nutrients, etc. to synthesize specific signal molecules and excrete/absorb the molecules to and from outside, thereby perceiving the peripheral cell density. When the cell density increases and the concentration of the signal molecules reaches a threshold level, expression of specific genes begins. As a result, the group behavior of the microorganisms is regulated and this phenomenon is called quorum sensing. Generally, the quorum sensing occurs in environments where the cell density is high. As representative examples of the quorum sensing phenomenon, symbiosis, virulence, competition, conjugation, antibiotic production, motility, sporulation and biofilm formation have been reported (Fuqua et al., Ann. Rev. Microbiol., 2001, Vol. 50, pp. 725-751).

In particular, the quorum sensing mechanism of microorganisms may occur more frequently and easily in the case of a biofilm state with a remarkably higher cell density than in the case of a suspended state. Davies et al. reported in 1998 that the quorum sensing mechanism of the pathogen Pseudomonas aeruginosa is closely related to various characteristics of biofilm including the extent of biofilm formation, its physical and structural properties such as thickness and morphology, antibiotic resistance of the microorganism, or the like (Science, Vol. 280, pp. 295-298). Since then, researches for inhibiting biofilm formation by artificial regulation of the quorum sensing mechanism have been made in the field of medicine and agriculture so as to prevent contamination of medical appliances (Baveja et al., Biomaterials, 2004, Vol. 50, pp. 5003-5012) or to control plant diseases (Dong et al., Nature, 2001, Vol. 411, pp. 813-817).

The conventional methods for inhibiting biofilm formation by regulating the quorum sensing mechanism of microorganisms are classified into several categories as follows.

Firstly, the biofilm formation can be inhibited by injecting an antagonist known to have a structure similar to that of a signal molecule used in the quorum sensing mechanism and compete with the signal molecule for a gene expression site. As representative antagonists, furanone secreted by Delisea pulchra, which is a species of red algae, and halogenated derivatives thereof have been reported (Henzer et al., EMBO Journal, Vol. 22, 3803-3815).

Secondly, the biofilm formation can be inhibited by an enzyme that decomposes a signal molecule used in the quorum sensing mechanism (enzyme that inhibits biofilm formation such as one that quenches quorum sensing of microorganisms; e.g., lactonase or acylase). For example, Xu et al. developed in 2004 a method for inhibiting biofilm formation on various surfaces by injecting a solution of the enzyme acylase that decomposes acyl-homoserine lactone (AHL) which is a signal molecule of Gram-negative bacteria (U.S. Pat. No. 6,777,223). The reaction whereby the signal molecule is decomposed by lactonase or acylase is as follows.

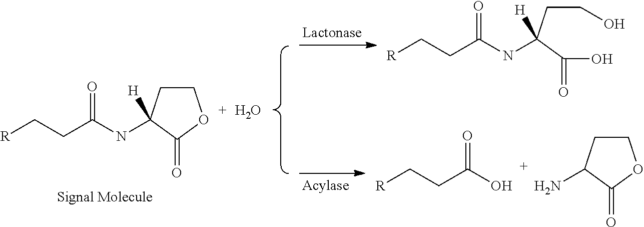

However, the method of inhibiting biofilm formation by directly injecting a solution of an enzyme for inhibiting quorum sensing is not practically applicable due to excessive loss of the enzyme and fast inactivation of the enzyme through denaturation.

As another method, a method of inhibiting biofouling on membrane surface of a submerged membrane bioreactor (sMBR) by immobilizing an enzyme for inhibiting quorum sensing (acylase) on a magnetic carrier by a layer-by-layer method, thereby preventing inactivation of the enzyme by denaturation and allowing easy separation and recovery of the enzyme-immobilized magnetic carrier using magnetic field has been reported recently (Korean Patent No. 981519). However, since microbial flocs are present at high concentrations and the flocs are taken out periodically to keep sludge retention time constant during the MBR process, there is a limit in completely recovering the magnetic carrier mixed with the flocs only through the magnetic field application. Also, in order to maximize the recovery rate of the magnetic carrier, a submerged type reactor wherein the carrier exists only in the reactor and does not circulate through the other interior parts of the system (e.g., tubing, valve, fitting, etc.) should be required. Accordingly, this method is inapplicable to high pressure membrane processes such as nanofiltration or reverse osmosis membrane processes most of which use external pressure-driven type reactors. In addition, since the method using the enzyme-immobilized magnetic carrier requires production of the enzyme through recombination of microorganisms involving culturing, extraction and purification of microorganisms to obtain the immobilizable enzyme, the production cost is high. Further, the immobilization of the purified enzyme by the layer-by-layer method requires a lot of time and cost.

The inventors of the present disclosure have researched to realize an economical and stable membrane water treatment process by applying a container in which, instead of biofilm formation-inhibiting enzymes, biofilm formation-inhibiting microorganisms producing the enzymes are immobilized therein in a water treatment reactor, thereby solving the above-described problems occurring when the enzymes are directly immobilized and applying the technique of inhibiting biofilm formation from a molecular biological approach to the membrane water treatment process.

### DISCLOSURE

### Technical Problem

The present disclosure is directed to providing a technique for inhibiting or reducing membrane biofouling in a membrane water treatment process, not from a physical/chemical approach like the conventional backwashing or chemical cleaning but from a molecular biological approach based on the understanding of the biofilm formation mechanism for sufficiently inhibiting the formation of the biofilm and, optionally, for providing effect of a physically washing membrane.

### Technical Solution

The inventors of the present disclosure have found out that membrane biofouling can be effectively inhibited or reduced in view of the molecular biological or physical perspective by applying a container for inhibiting biofilm formation in which biofilm formation-inhibiting microorganisms are immobilized in a permeable container to a membrane water treatment process and thereby stably maintaining the activity of the biofilm formation-inhibiting microorganisms.

The present disclosure provides a container with biofilm formation-inhibiting microorganisms immobilized therein comprising a permeable container and biofilm formation-inhibiting microorganisms immobilized in the container. The present disclosure also provides a membrane water treatment apparatus including a reactor accommodating water to be treated, a membrane module for water treatment and the container with biofilm formation-inhibiting microorganisms immobilized therein placed in the reactor.

In the present disclosure, the permeable container may be any container that can isolate and dispose biofilm formation-inhibiting microorganisms at high density in the water treatment reactor and has adequate permeability so as to allow inflow and outflow of oxygen, nutrients, metabolites, etc. required for the growth and activation of the biofilm formation-inhibiting microorganisms, without particular limitation in material, shape, etc. For example, it may be a porous container having a predetermined pore size distribution (see Embodiment 1) or a fluidisable carrier having fluidisability through aeration such as a hydrogel (see Embodiment 2).

Embodiment 1 of the present disclosure relates to a container for inhibiting biofilm formation comprising a hollow porous container and biofilm formation-inhibiting microorganisms immobilized therein.

FIGS. 1a-1d show schematic diagrams and photographs of a container for inhibiting biofilm formation according to Embodiment 1 of the present disclosure (FIGS. 1a-1b: both ends sealed; FIGS. 1c-1d: one end sealed) and FIG. 3 shows a schematic diagram of a membrane bioreactor apparatus for water treatment in which the container for inhibiting biofilm formation is accommodated.

The container according to Embodiment 1 of the present disclosure may be prepared by capturing the biofilm formation-inhibiting microorganisms inside the hollow porous container. Since the biofilm formation-inhibiting microorganisms are immobilized in the hollow porous container, materials such as biofilm formation-inhibiting enzymes can be efficiently discharged toward the water treatment reactor without loss of the biofilm formation-inhibiting microorganisms toward the water treatment reactor. As a result, biofouling on the membrane surface and in the pores of the membrane can be reduced stably.

There is no particular limitation on the material or shape of the hollow porous container according to Embodiment 1 of the present disclosure as long as it has a porosity that allows the transfer of fine materials such as biofilm formation-inhibiting enzymes, water and signal molecules without loss of the biofilm formation-inhibiting microorganisms. For example, a hollow membrane of a tubular or hollow fiber type commonly used for water treatment or a filter container fabricated to a predetermined shape may be used.

Since most microorganisms are generally 1-10 μm in size on average, a hollow porous container having an average pore size smaller than this may be used to minimize the loss of the microorganisms.

The container for inhibiting biofilm formation according to Embodiment 1 of the present disclosure may be prepared by injecting/capturing the biofilm formation-inhibiting microorganisms inside the hollow porous container and sealing both ends (see FIGS. 1a and 1b). Alternatively, only one end may be sealed and the other end may be connected with a conduit to which a porous member for preventing outflow of the microorganisms such as a filter so as to be exposed to the atmosphere outside the water treatment reactor, such that mass transfer of water, biofilm formation-inhibiting enzymes, etc. through the hollow membrane in the water treatment reactor can be easier (see FIGS. 1c and 1d).

Meanwhile, Embodiment 2 of the present disclosure relates to a container for inhibiting biofilm formation comprising a permeable container (carrier) having fluidisability through submerged aeration and biofilm formation-inhibiting microorganisms immobilized in the container (carrier). Owing to the biofilm formation-inhibiting microorganisms immobilized in the carrier, biofilm formation can be inhibited molecular biologically. In addition, a biofilm formed on the membrane surface can be detached physically by direct application of physical impact derived from the fluidisability of the carrier under submerged aeration condition.

In Embodiment 2 of the present disclosure, the “immobilization” of biofilm formation-inhibiting microorganisms in a fluidisable carrier includes the adhesion/entrapment/encapsulation/collection/supporting of the biofilm formation-inhibiting microorganisms in the inside space of the matrix of the fluidisable carrier.

Particular shapes or materials of the permeable carrier in Embodiment 2 are not specifically limited in view of the mechanical strength, flexibility, etc. only if mass transfer across the carrier surface is possible, and the surface of the membrane is not damaged even by the contact with the membrane surface under submerged aeration conditions. Thus, a somewhat rigid shape or material may be used, and a flexible shape or material which may be freely bent and have resilience force in water flow may be used. The flexible shape or material may be used to minimize the damage of the membrane surface under stronger aeration conditions while maximizing the physical detachment of the biofilm.

More particularly, in Embodiment 2 of the present disclosure, the permeable carrier may include as a main component a hydrogel comprising hydrophilic polymers. More specifically, the hydrogel may include at least one polymer selected from a group consisting of alginate-based, PVA-based, polyethylene glycol-based and polyurethane-based (or composites thereof). As a result, mass transfer into and out of the fluidisable carrier can become easier and damage to the membrane surface due to the contact with the membrane surface even under strong submerged aeration condition can be prevented.

In addition to a hydrophilic polymer, the permeable carrier of Embodiment 2 may comprise a carbonaceous additive such as graphene oxide (GO) and carbon nanotubes (CNT) to increase mechanical strength, and/or a bio-inspired adhesive polymer additives such as a polydopamine polymer and a polynorepinephrine polymer to increase internal adhesiveness.

The hydrogel in Embodiment 2 of the present disclosure may have a 3-dimensional network structure through internal chemical crosslinking, such that the biofilm formation-inhibiting microorganisms can be captured therein and grow inside the carrier.

For example, alginate-based polymer is a typical hydrophilic polymer material possibly used as a natural carrier. In calcium chloride solution, this material forms a solid with a network structure through chemical crosslinking, which minimizes resistance to mass transfer. Therefore, it can immobilize not only the biofilm formation-inhibiting microorganisms but also the enzymes produced by the microorganisms. It is also advantageous in that it is suitable because of superior biocompatibility to be used in a reactor where microorganisms for water treatment, responsible for biofilm formation, exist and is unharmful to the human body while being inexpensive and economical.

Further, the form or geometry of the permeable carrier according to Embodiment 2 of the present disclosure is not specifically limited only if the damage onto the surface of an submerged-type membrane under aeration conditions may be prevented, and the biofilm formation-inhibiting microorganisms in the carrier may make contact with external water to be treated. Certain forms of carriers including substantially spherical or close thereto may be used. Further, various modified forms of carriers including columnar or sheet-like to increase the surface area of the carrier for the contact with the water to be treated and the membrane surface.

The fluidisable carrier with substantially spherical form or close thereto may effectively induce the removal of the biofilm on the membrane surface by disposing the biofilm formation-inhibiting microorganisms in a bulky, substantially spherical and fluidisable carrier (as a matrix), forming a container with biofilm formation-inhibiting microorganisms immobilized therein, and applying physical blow easily to the surface of the membrane with an appropriate force through the movement of the substantially spherical carrier under submerged aeration.

In the columnar fluidisable carrier as a modification embodiment of Embodiment 2 for increasing substantial area (particularly, surface area per carrier volume) for contact of biofilm formation-inhibiting microorganisms with water to be treated and a membrane surface, the mass transfer across the surface of the carrier may be improved more efficiently so as to enhance effect of the inhibition for the biofilm-formation by disposing the biofilm formation-inhibiting microorganisms in the long columnar fluidisable carrier (as a matrix) to form a container with the biofilm formation-inhibiting microorganisms immobilized therein, and the biofilm on the surface of the membrane may be readily detached under submerged aeration by the fluidisability of the carrier with the increased area for contact of the fluidisable carrier with the membrane surface, thereby reducing membrane fouling in a membrane water treatment process. Here, the “columnar” fluidisable carrier may include not only a hard carrier of which central axis is linear or close thereto but also a crooked carrier of which central axis is curved. Further, it may include not only a circular columnar carrier having a circular cross-section perpendicular to the central axis thereof, but also a polygonal columnar carrier having a polygonal cross-section. Furthermore, the circular columnar carrier may include not only a carrier of which cross-section perpendicular to the central axis thereof is a true circle but also a carrier of which cross-section is an ellipse or a close form thereof.

As for particular structures of the columnar fluidisable carrier, it may include a columnar fluidisable carrier of which inner cross-section is fully filled (non-hollow) and a columnar fluidisable carrier of which at least a portion of the inner cross-section is empty (hollow). Since the hollow columnar fluidisable carrier may have additional surface area per carrier volume, the biofilm formation-inhibiting microorganisms therein may make further contact with external water to be treated across inner surface in a length direction (lumen side), in addition to the outer surface in the length side (shell side), and accomplish additional effect of biofilm formation-inhibition.

The dimension of the columnar fluidisable carrier is not specifically limited only if the length is sufficient relative to the diameter of the cross-section to secure a sufficient area for contact with water to be treated and a membrane surface, however a columnar fluidisable carrier having an aspect ratio, which is a ratio of the maximum diameter of the cross-section (corresponding to a diameter in case of the cross-section of a true circle) to the length of the column, of particularly around 5-500, and more particularly around 20-100 may be used. If the aspect ratio is too small, the preparation of the columnar carrier is difficult, and the accomplishment of the maximization of the substantial area (surface area per carrier volume) for contact with the external water to be treated and the membrane surface may be difficult. If the aspect ratio is too large, too long columnar carrier may be entangled in the water to be treated, and the mass transfer of important materials such as biofilm formation-inhibiting enzymes may be deteriorated. The diameter and the length of the columnar fluidisable carrier is not specifically limited, however the diameter of the cross-section may be around 0.2-20 mm, and the length may be around 1-1,000 mm. For the hollow columnar fluidisable carrier, the outer diameter of the cross-section may be around 0.2-20 mm, the inner diameter of the cross-section may be around 0.1-10 mm, and the length may be around 1-1,000 mm.

In a sheet-like fluidisable carrier as another modification of Embodiment 2 of the present disclosure for increasing substantial area for contact with water to be treated and a membrane surface (see FIG. 22c), since biofilm formation-inhibiting microorganisms are disposed in a fluidisable carrier (as a matrix) with a planar form to make a container with the biofilm formation-inhibiting microorganisms immobilized therein, a substantial area for contact with water to be treated is further increased. Further, since solid physical shape of the container can be easily maintained, the sheet-like fluidisable carrier can substantially secures larger substantial area for contact with a membrane surface, to increase the opportunity for detachment of the biofilm on the membrane surface. Furthermore, entanglement phenomenon of the fluidisable carriers may be restrained, and problems of loss of fluidisability due to the trapping of the fluidisable carrier between individual hollow fibers in a hollow fiber membrane module of a membrane water treatment apparatus may be largely decreased, thereby achieving more efficient inhibition of the biofilm formation.

The dimension of the sheet-like fluidisable carrier of the present disclosure is not specifically limited only if it has a sufficiently large surface area to volume (SA/V) obtained by dividing total surface area by carrier volume to secure sufficient area for contact with the water to be treated and the membrane surface. A sheet-like carrier having the ratio of SAN of particularly around 5-1,000 mm⁻¹, and more particularly, around 10-100 mm⁻¹ may be used. If the SAN ratio is too small, sufficient area for contact with external water to be treated and the membrane surface is not secured, and the improvement of water permeability due to the biofilm formation-inhibiting mechanism and the physical removal (detachment) of the biofilm on the membrane surface may be deteriorated. If the SAN ratio is too large, the thickness of the carrier may be too small, and the physical strength of the carrier may be largely decreased. The surface area and the average thickness of the sheet-like fluidisable carrier are not specifically limited, however the surface area may be around 1-200 cm², and more particularly, around 2-100 cm², and the average thickness may be around 0.1-5 mm, and more particularly, around 0.2-2 mm.

Since the size of the fluidisable carrier of Embodiment 2 of the present disclosure is easily controllable, the carrier can be easily separated and recovered using means such as microsieves and a screen. Accordingly, the recovery problem of the conventional magnetic carrier container can be solved.

The biofilm formation-inhibiting microorganisms that can be used in the present disclosure may be any recombined or natural microorganisms capable of producing enzymes for inhibiting biofilm formation. Representatively, microorganisms capable of producing enzymes for inhibiting quorum sensing that decompose signal molecules used in the quorum sensing mechanism may be used. Specifically, microorganisms producing enzymes for inhibiting quorum sensing such as acylase or lactonase that is enzyme for decomposing signal molecules (AHL) of gram negative bacteria may be used. For example, E. coli obtained by genetically recombining E. coli XL1-blue with the aliA gene (which is involved in the production of lactonase) extracted from Bacillus thuringiensis subsp. kurstaki or naturally occurring microorganisms (e.g., Rhodococcus qingshengii bacteria) may be used.

Meanwhile, when certain bacteria (including bacteria for water treatment) present in water are contacted with farnesol which is secreted from certain fungi and known as signal molecules for quorum sensing mechanism of the fungi, the formation of the biofilm may be restrained by inhibiting the quorum sensing mechanism of the bacteria (Gomes et al., Curr Microbiol (2009) 59: 118-122). According to the additional researches by the present inventors, it is supposed that the farnesol is involved in the inhibition of the quorum sensing mechanism by AutoInducer-2 (AI-2), which is a signal molecule commonly used by the bacteria in water, i.e., gram negative (bacteria) and gram positive (bacteria). In the present disclosure, the biofilm formation-inhibition microorganisms such as fungi in Candida genus, more particularly, Candida albicans that may produce the substance for inhibiting quorum sensing of the bacteria in water, i.e., the farnesol, may be used. Since fungal microorganisms have superior environmental adaptability including climate-resistance relative to bacteria microorganisms, they may have an additional advantage that the inhibition effect of the biofilm formation under severe environmental conditions, as in the inside of the membrane bioreactor, may be maximized.

In order to acquire bacteria of species Rhodococcus qingshengii suitable to be used for a water treatment process in an embodiment of the present disclosure, some microorganisms were isolated from sludge obtained from the bioreactors of municipal wastewater treatment plants and separated, from the isolated microorganisms, and the bacteria of the genus Rhodococcus (including Rhodococcus qingshengii) with excellent activity of decomposing AHL signal molecules were isolated through enrichment culture method. In another embodiment of the present disclosure, genetically recombined fungi of Candida albicans, which was modified to secrete excessive amount of farnesol that has activity for inhibiting AI-2 quorum sensing, was used.

There is no particular limitation on the method of immobilizing the biofilm formation-inhibiting microorganisms inside the fluidisable carrier. In addition to adhesion, entrapment, encapsulation, supporting, etc. a method of simply injecting the microorganisms into the container and capturing them may also be used.

In Embodiment 1 of the present disclosure, the biofilm formation-inhibiting microorganisms are injected into the hollow porous container such as a membrane using a pump (see FIG. 2).

Meanwhile, in some examples of Embodiment 2 of the present disclosure, a suspension wherein the biofilm formation-inhibiting microorganisms are suspended in water at high concentration is mixed with a hydrogel, etc. to prepare a carrier solution, and the carrier solution is added to a calcium chloride solution (crosslinking solution) at a predetermined rate using a peristaltic pump such that the microorganisms are ‘entrapped’ to prepare a spherical fluidisable carrier of a predetermined size with biofilm formation-inhibiting microorganisms immobilized therein and having (see FIG. 11). In addition, in another examples of Embodiment 2 of the present disclosure, a non-hollow circular columnar fluidisable carrier with biofilm formation-inhibiting microorganisms immobilized therein may be prepared by discharging a carrier solution comprising a microorganism suspension into a calcium chloride solution (crosslinking solution) using a syringe or a syringe pump, or a hollow circular columnar fluidisable carrier may be prepared by passing a solvent through an inner tube and discharging a carrier solution through an outer tube by means of a double pipe nozzle (see FIGS. 22a and 22b). Furthermore, a sheet-like fluidisable carrier as another modification of Embodiment 2 of the present disclosure may be prepared, similarly as in the columnar fluidisable carrier, by mixing a high concentration microorganism suspension in which biofilm formation-inhibiting microorganisms are suspended in water with hydrogel, etc. to prepare a carrier solution, coating the carrier solution on a planar surface such as a planar glass plate to a uniform thickness using a casting knife, and immersing into a calcium chloride solution (crosslinking solution) (see FIG. 22c).

The present disclosure also provides a membrane water treatment apparatus including a water treatment reactor wherein the container for inhibiting biofilm formation is disposed and a membrane module for water treatment. The membrane module that can be used in the membrane water treatment apparatus of the present disclosure may be any general membrane module for water treatment capable of achieving improved permeability by inhibiting or reducing membrane biofouling and is not particularly limited. Further, the membrane water treatment apparatus of the present disclosure may be not only the general membrane water treatment apparatus such as microfiltration membrane apparatus or ultrafiltration membrane apparatus but also the advanced water treatment apparatus such as nanofiltration apparatus and reverse osmosis apparatus wherein a biofilm is formed on the membrane surface by microorganisms existing in the water to be treated in addition to the membrane bioreactor (MBR) apparatus wherein a biofilm is formed on the membrane surface by various microorganisms used for water treatment.

### Advantageous Effects

When applied to an actual membrane water treatment process, the container for inhibiting biofilm formation of the present disclosure can inhibit the formation of biofilms on the membrane surface and, optionally, can provide an effect of physically washing membrane. As a result, decrease of permeability can be prevented, membrane cleaning cycle is lengthened, consumption of cleansers can be reduced, and long-term membrane filtration can be conducted. Particularly, in the columnar or sheet-like permeable carrier which has larger surface area per carrier volume, biofilm formation-inhibiting microorganisms may be immobilized in the carrier (as a matrix). Thus, mass transfer across the carrier surface may be more efficient, and the formation of the biofilm may be effectively inhibited in the view of molecular biological perspective. Further, the carrier is not readily trapped in a certain type of membrane module, and the detachment of the biofilm owing to physical blow onto the membrane surface may be more effectively induced since a sufficient area for contact with membrane surface may be secured, thereby maximizing effects of inhibiting/removing membrane fouling.

### DESCRIPTION OF DRAWINGS

FIGS. 1a-1d show schematic diagrams and photographs of a container for inhibiting biofilm formation with biofilm formation-inhibiting microorganisms immobilized therein according to Embodiment 1 of the present disclosure (FIGS. 1a-1b: both ends sealed; FIGS. 1c-1d: one end sealed).

FIG. 2 schematically shows a process of preparing the container for inhibiting biofilm formation according to Embodiment 1 of the present disclosure.

FIG. 3 shows a schematic diagram of a membrane bioreactor process using a membrane bioreactor apparatus for water treatment accommodating the container for inhibiting biofilm formation according to Embodiment 1 of the present disclosure.

FIG. 4 shows increase of transmembrane pressure (increase of membrane biofouling) in Example 2A according to Embodiment 1 of the present disclosure and in Comparative Example 2A.

FIG. 5 shows signal molecule decomposition activity of the container for inhibiting biofilm formation according to Embodiment 1 of the present disclosure.

FIG. 6 shows that the signal molecule decomposition activity of the container for inhibiting biofilm formation according to Embodiment 1 of the present disclosure is maintained for a long period of time.

FIG. 7 shows increase of transmembrane pressure (increase of membrane biofouling) in Example 4A according to Embodiment 1 of the present disclosure and in Comparative Example 4A.

FIG. 8 shows increase of transmembrane pressure (increase of membrane biofouling) in Example 5A according to Embodiment 1 of the present disclosure and in Comparative Example 5A.

FIGS. 9a and 9b show a schematic diagram of a container (comprising spherical fluidisable carrier) for inhibiting biofilm formation with biofilm formation-inhibiting microorganisms immobilized therein according to Embodiment 2 of the present disclosure and photographs of a realistically prepared container (fluidisable carrier).

FIGS. 10a and 10b show photographs of a bioreactor including the container for inhibiting biofilm formation according to Embodiment 2 of the present disclosure (FIG. 10a: without aeration; FIG. 10b: with aeration).

FIG. 11 shows a process of preparing a container for inhibiting biofilm formation according to Embodiment 2 of the present disclosure.

FIG. 12 shows signal molecule decomposition activity of a container for inhibiting biofilm formation according to Embodiment 2 of the present disclosure.

FIG. 13 shows a schematic diagram of a membrane bioreactor apparatus accommodating the container for inhibiting biofilm formation according to Embodiment 2 of the present disclosure.

FIG. 14 shows increase of transmembrane pressure (increase of membrane biofouling) versus operation time, in Example 1B according to Embodiment 2 of the present disclosure and in Comparative Examples 1B and 2B.

FIG. 15 shows signal molecule decomposition activity (relative activity) of a fluidisable spherical carrier versus operation time, according to Embodiment 2 of the present disclosure in a membrane bioreactor apparatus.

FIG. 16 shows the degree of growth of biofilm formation-inhibiting microorganisms inside a spherical fluidisable carrier (as wet weight of the fluidisable carrier) versus operation time, according to Embodiment 2 of the present disclosure of a membrane bioreactor apparatus.

FIG. 17 shows an embodiment of preparing a container (comprising a spherical fluidisable carrier) with biofilm formation-inhibiting microorganisms (fungi) immobilized therein according to Embodiment 2 of the present disclosure.

FIG. 18 shows the assessment results of the activity for inhibiting quorum sensing of a container (comprising a spherical fluidisable carrier) with biofilm formation-inhibiting microorganisms (fungi) immobilized therein according to Embodiment 2 of the present disclosure.

FIG. 19 shows a schematic diagram of a membrane bioreactor apparatus for accommodating a container (comprising a spherical fluidisable carrier) with biofilm formation-inhibiting microorganisms (fungi) immobilized therein according to Embodiment 2 of the present disclosure in a bioreactor.

FIG. 20 shows increase of transmembrane pressure versus operation time in membrane bioreactor apparatuses, accommodating containers (comprising a spherical fluidisable carrier) with biofilm formation-inhibiting microorganisms (fungi) immobilized therein according to Example and Comparative Example of Embodiment 2 of the present disclosure.

FIGS. 21a and 21b show schematic diagrams of containers (comprising non-hollow and hollow circular columnar fluidisable carrier, respectively) with biofilm formation-inhibiting microorganisms immobilized therein according to Embodiment 2 of the present disclosure, and FIG. 2c shows a schematic diagram of a container (comprising sheet-like fluidisable carrier) with biofilm formation-inhibiting microorganisms immobilized therein according to Embodiment 2 of the present disclosure.

FIGS. 22a and 22b show an embodiment of preparing containers (comprising non-hollow or hollow circular columnar fluidisable carrier, respectively) with biofilm formation-inhibiting microorganisms immobilized therein according to an Embodiment 2 of the present disclosure, and FIG. 22c shows an embodiment of preparing a container with biofilm formation-inhibiting microorganisms immobilized therein (comprising sheet-like fluidisable carrier) according to Embodiment 2 of the present disclosure.

FIGS. 23a and 23b show photographs taken on containers (comprising non-hollow or hollow circular columnar fluidisable carrier, respectively) with biofilm formation-inhibiting microorganisms immobilized therein according to an Embodiment 2 of the present disclosure, and FIG. 23c shows a photograph taken on a container (comprising sheet-like fluidisable carrier) with biofilm formation-inhibiting microorganisms immobilized therein according to Embodiment 2 of the present disclosure.

FIG. 24 shows the assessment results of the decomposition activity of signal molecules of a container (comprising non-hollow circular columnar fluidisable carrier) with biofilm formation-inhibiting microorganisms immobilized therein according to Embodiment 2 of the present disclosure.

FIG. 25 shows the assessment results of the decomposition activity of signal molecules of a container (comprising hollow circular columnar fluidisable carrier) with biofilm formation-inhibiting microorganisms immobilized therein according to Embodiment 2 of the present disclosure.

FIG. 26 shows a schematic diagram of a membrane bioreactor apparatus for accommodating containers (comprising non-hollow circular columnar fluidisable carrier) with biofilm formation-inhibiting microorganisms immobilized therein according to Embodiment 2 of the present disclosure and operating.

FIG. 27 shows increase of transmembrane pressure versus operation time in membrane bioreactor apparatuses, accommodating containers (comprising non-hollow circular columnar fluidisable carrier) with biofilm formation-inhibiting microorganisms immobilized therein according to Example and Comparative Example of Embodiment 2 of the present disclosure.

FIG. 28 shows increase of transmembrane pressure versus operation time in a membrane bioreactor apparatus, accommodating containers (comprising spherical fluidisable carrier and a non-hollow circular columnar fluidisable carrier, respectively) with biofilm formation-inhibiting microorganisms immobilized therein according to Examples of Embodiment 2 of the present disclosure.

FIG. 29 shows a schematic diagram of a membrane bioreactor apparatus for accommodating containers (comprising hollow circular columnar fluidisable carrier) with biofilm formation-inhibiting microorganisms immobilized therein according to Embodiment 2 of the present disclosure.

FIG. 30 shows increase of transmembrane pressure versus operation time in membrane bioreactor apparatuses, accommodating containers (comprising hollow circular columnar fluidisable carrier) with biofilm formation-inhibiting microorganisms immobilized therein according to Example and Comparative Example of Embodiment 2 of the present disclosure.

### MODE FOR INVENTION

Hereinafter, the present disclosure will be described in detail through examples. However, the present disclosure is not limited thereto.

Embodiment 1—Hollow porous container with biofilm formation-inhibiting microorganisms (genetically recombined microorganisms) immobilized therein

### PREPARATION EXAMPLE 1A

### Preparation of a Container with Biofilm Formation-Inhibiting Microorganisms Immobilized Therein (Both Ends Sealed)

Genetically recombined E. coli capable of producing lactonase was used as biofilm formation-inhibiting microorganisms. Specifically, E. coli XL1-blue, which is commonly used in genetic recombination, was used and the aiiA gene from the Bacillus thuringiensis subsp. kurstaki was inserted therein through genetic recombination. The aiiA gene codes for lactonase which decomposes signal molecules used in the quorum sensing mechanism.

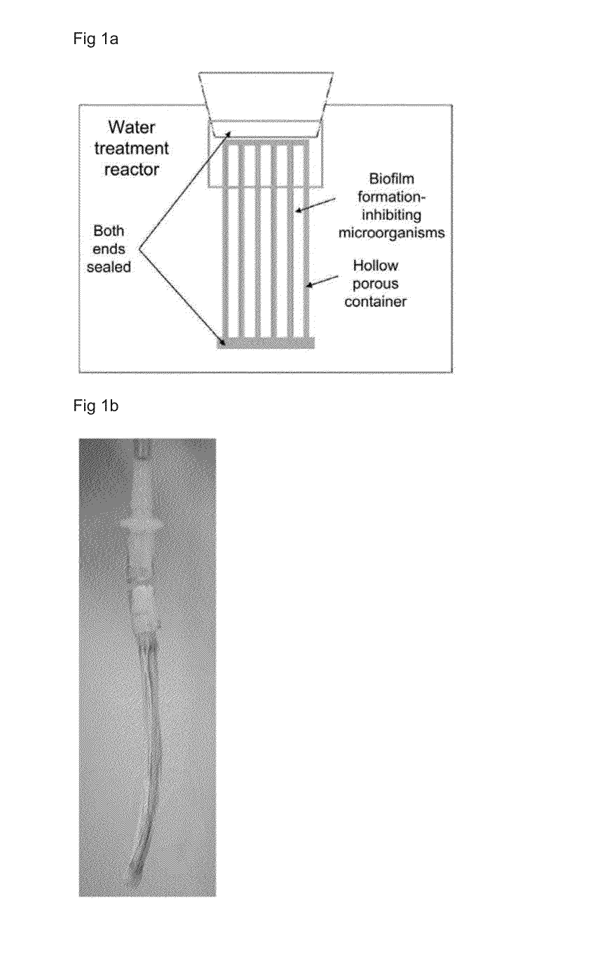

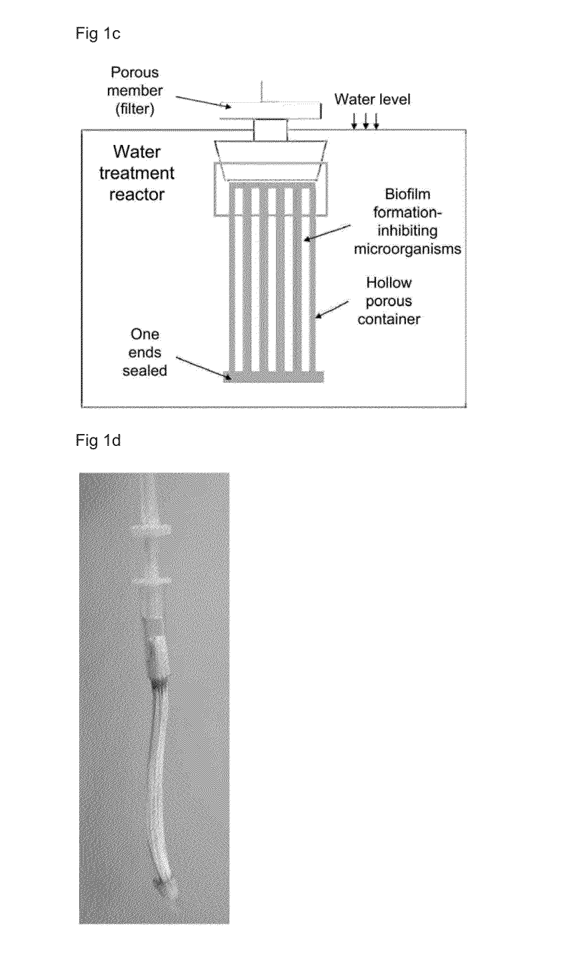

As a hollow porous container for immobilizing the biofilm formation-inhibiting microorganisms, a hollow fiber membrane (available from Econity Co., Ltd) was used. Since the hollow fiber membrane has a pore size of 0.4 μm, the microorganisms cannot pass therethrough whereas water and signal molecules can easily pass therethrough and travel between the container and a reactor. A total of 55 strands of hollow fiber membranes were used to prepare a container with biofilm formation-inhibiting microorganisms immobilized therein having a length of 10 cm and a total membrane surface area of 112.31 cm², with both ends sealed, as shown in FIGS. 1 a and 1 b.

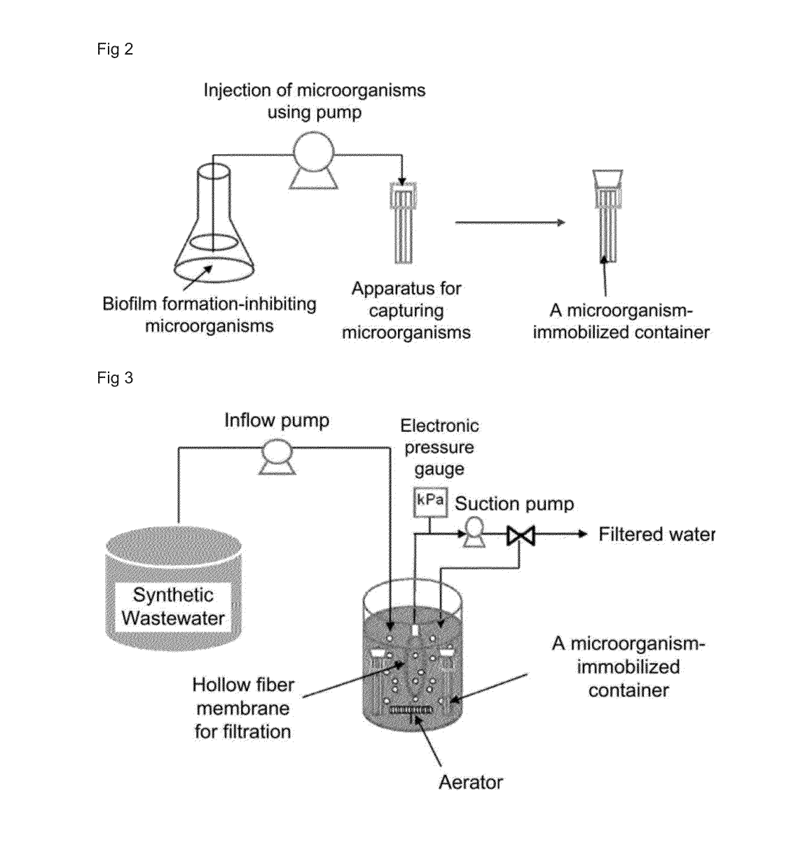

After culturing for 24 hours, 200 mL of E. coli was centrifuged and the supernatant was discarded thereby removing the culture medium. The microorganisms were resuspended using Tris-HCl 50 mM buffer (pH 7.0) and then injected into the container using a pump, as shown in FIG. 2.

### PREPARATION EXAMPLE 2A

### Preparation of a Container with Biofilm Formation-Inhibiting Microorganisms Immobilized Therein (One End Sealed)

A container with biofilm formation-inhibiting microorganisms immobilized therein was prepared in the same manner as in Preparation Example 1A, except that only one end of the container submerged in a reactor was sealed and the other end was communicated with the outside atmosphere via a filter member (PTFE, pore size 0.45 μm) followed by a tube, and then biofilm formation-inhibiting microorganisms (E. coli ) were injected (see FIGS. 1c, 1c, 1d and 2).

### EXAMPLE 1A

### Measurement of Signal Molecule Decomposition Activity of a Container with Biofilm Formation-Inhibiting Microorganisms Immobilized Therein

Signal molecule (AHL) decomposition activity of the container with biofilm formation-inhibiting microorganisms immobilized therein was measured using N-octanoyl-L-homoserine lactone (OHL), which is one of representative signal molecules. After adding Tris-HCl 50 mM buffer (pH 7) to a test tube and then injecting OHL to a concentration of 0.2 μM, the container with biofilm formation-inhibiting microorganisms immobilized therein was added thereto and the resulting mixture was reacted for 90 minutes in a shaking incubator of 30° C. at 200 rpm. As a result, about 60% of signal molecules were decomposed for 90 minutes (see FIG. 5).

### COMPARATIVE EXAMPLE 1A

The same procedure was repeated as Example 1A except that the microorganisms were not injected to the container. As a result, the signal molecules were hardly decomposed (see FIG. 5).

### EXAMPLE 2A

### Application to Membrane Bioreactor Process (Genetically Recombined Microorganisms/Both Ends-Sealed Container)

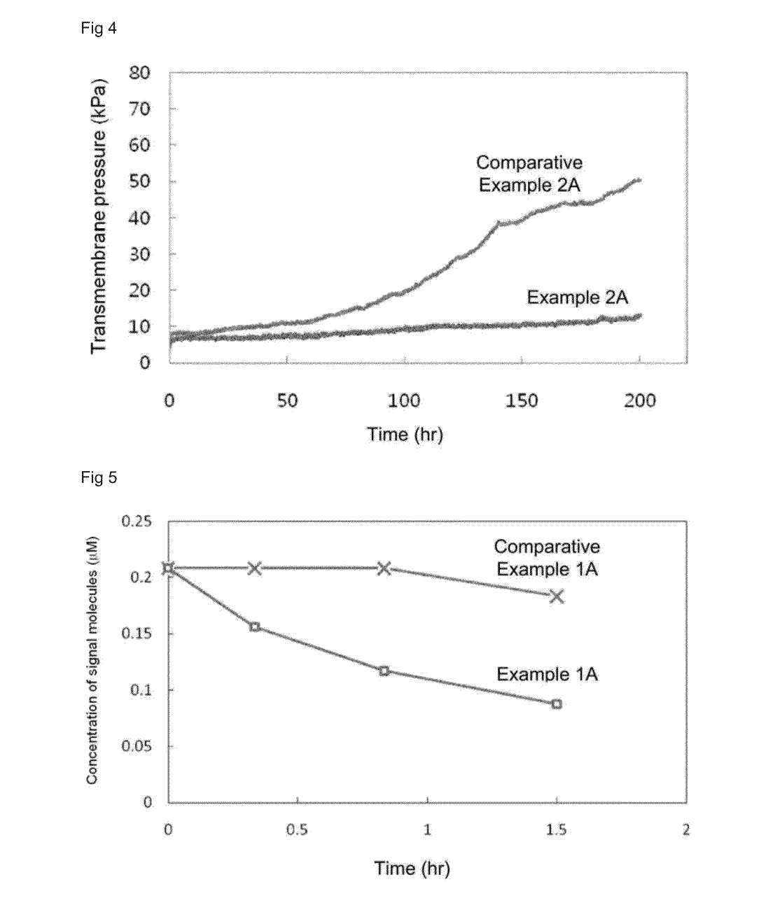

The container with biofilm formation-inhibiting microorganisms immobilized therein, prepared in Preparation Example 1A, was applied to a laboratory-scale membrane bioreactor process (see FIG. 3). Specifically, 1.2 L of activated sludge was filled in a cylindrical reactor and diffuser stone was equipped at the bottom to maintain aeration of 1 L/min. A total of two pieces of containers with biofilm formation-inhibiting microorganisms immobilized therein were placed in the reactor symmetrically. For continuous operation, synthetic wastewater containing glucose as a main carbon source was introduced by an inflow pump. The chemical oxygen demand (COD) of the synthetic wastewater was about 550 ppm and hydraulic retention time was about 12 hours. The synthetic wastewater was filtered with a flux of about 18 L/m²·hr through a hollow fiber ultrafiltration membrane (Zeeweed 500, GE-Zenon, pore size 0.04 μm) submerged in the reactor. The water level of the reactor was maintained by recirculating a part of the permeate using a level controller and a 3-way-valve. During the operation, mixed liquor suspended solids (MLSS) was maintained at 4500-5000 mg/L. The degree of membrane biofouling caused by biofilm formation on the membrane surface was represented with transmembrane pressure (TMP). The higher the transmembrane pressure, the larger is the degree of membrane biofouling. Even after operation for about 200 hours, the transmembrane pressure was no more than about 13 kPa (see FIG. 4).

### COMPARATIVE EXAMPLE 2A

The same procedure was repeated as Example 2A except that the microorganisms were not injected to the container. After operation for about 200 hours, the transmembrane pressure reached about 50 kPa (see FIG. 4).

### EXAMPLE 3A

### Maintenance of Activity of a Container with Biofilm Formation-Inhibiting Microorganisms Immobilized Therein

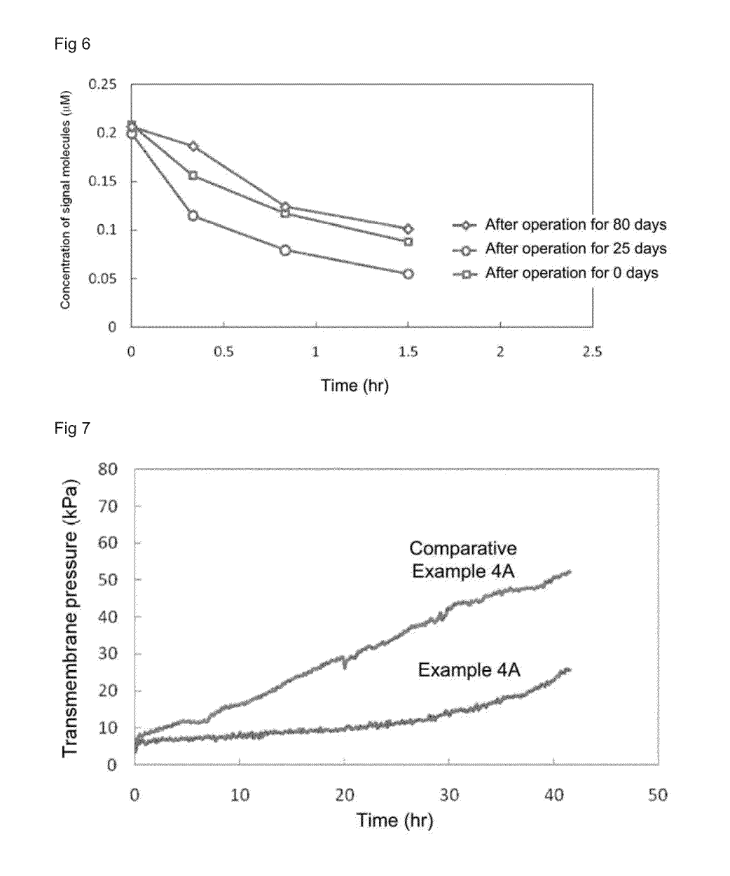

It was investigated whether the signal molecule decomposition activity of the container with biofilm formation-inhibiting microorganisms immobilized therein is maintained for a long period of time. Specifically, after continuous operation for 25 days and 80 days, the container with biofilm formation-inhibiting microorganisms immobilized therein was taken out from the reactor and, followed by washing the outside of the container several times with distilled water, the same procedure as Example 1A was conducted (see FIG. 6). Even after operation for 80 days, the signal molecule decomposition activity was not significantly decreased.

### EXAMPLE 4A

### Application to Membrane Bioreactor Process (Natural Microorganisms/Both Ends-Sealed Container)

The microorganisms used in Example 2A were genetically modified by inserting the lactonase-producing gene into E. coli and they cannot survive in the actual wastewater environment for a long period of time. Therefore, in order to find microorganisms suitable to be applied to the actual water treatment process, microorganisms were isolated from sludge obtained from a sewage disposal plant located in Okcheon, Chungchengbuk-do, Korea. From the isolated microorganisms, the microorganisms of the genus Rhodococcus with excellent activity of decomposing signal molecules could be separated through enrichment culture. A container with biofilm formation-inhibiting microorganisms immobilized therein was prepared using these microorganisms, in the same manner as in Preparation Example 1A, and it was applied to a membrane bioreactor process under the same condition as Example 2A.

The container with biofilm formation-inhibiting microorganisms immobilized therein prepared above was applied to a laboratory-scale membrane bioreactor process. After operation for about 40 hours, transmembrane pressure reached about 24 kPa (see FIG. 7).

### COMPARATIVE EXAMPLE 4A

The same procedure was repeated as Example 4A except that the microorganisms were not injected to the container. After operation for about 40 hours, the transmembrane pressure reached about 50 kPa (see FIG. 7).

### EXAMPLE 5A

### Application to Membrane Bioreactor Process (Natural Microorganisms/One End-Sealed Container)

A membrane bioreactor was operated under the same condition as Example 4A, except that 2.5 L of activated sludge used in Example 4A was filled in a cylindrical reactor, a total of four pieces of containers with biofilm formation-inhibiting microorganisms immobilized therein were placed in the reactor symmetrically, hydraulic retention time of glucose-containing synthetic wastewater was set to about 8 hours, the flux of the wastewater through the membrane was changed to about 30 L/m²·hr and MLSS was maintained at 7500-8500 mg/L.

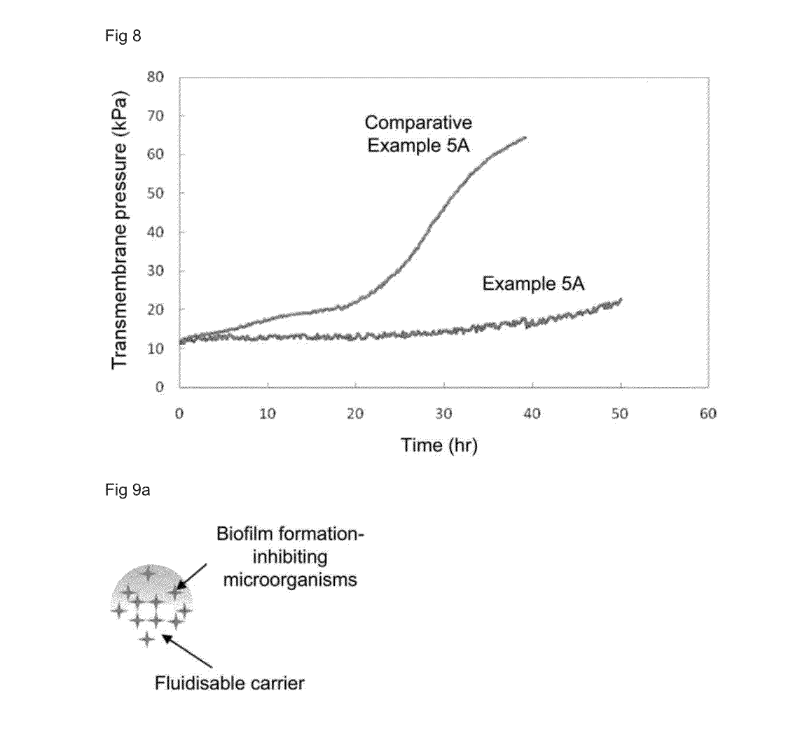

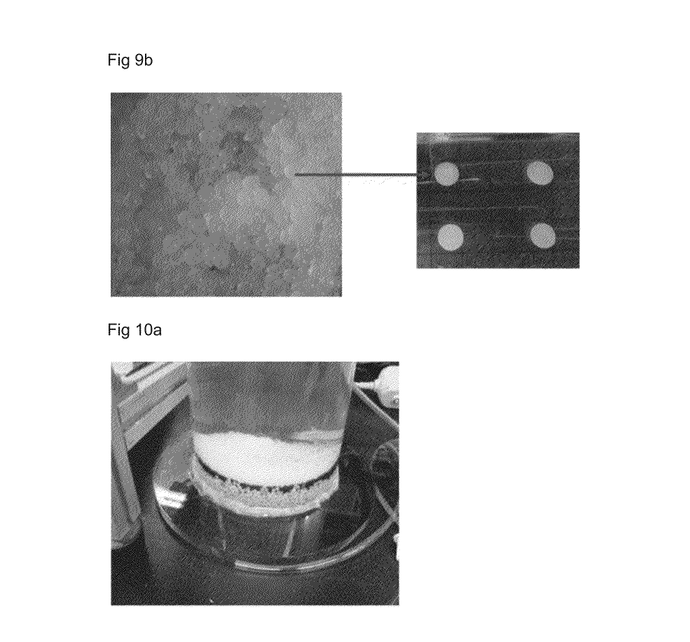

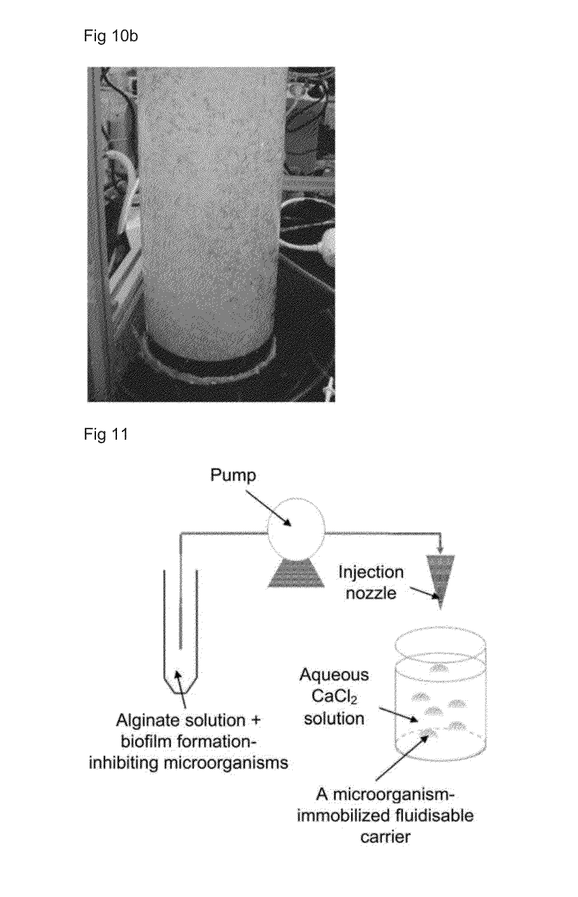

After operation for about 50 hours, the transmembrane pressure reached about 22 kPa (see FIG. 8).

### COMPARATIVE EXAMPLE 5A

The same procedure was repeated as Example 5A except that the microorganisms were not injected to the container. After operation for about 40 hours, the transmembrane pressure reached about 64 kPa (see FIG. 8).

### Embodiment 2

### 1. Embodiment 2 Concerning Spherical Fluidisable Carrier with Biofilm Formation-Inhibiting Microorganisms (Bacteria) Immobilized Therein

### PREPARATION EXAMPLE 1B

### Preparation of a Spherical Fluidisable Carrier with Biofilm Formation-Inhibiting Microorganism Immobilized Therein and Measurement of Signal Molecule Decomposition Activity

As biofilm formation-inhibiting microorganisms, Rhodococcus qingshengii BH4, known to produce lactonase which is one of enzymes for inhibiting quorum sensing, that was isolated from sludge from the municipal wastewater treatment plant in the same manner described in Embodiment 1 was used.

As a spherical fluidisable carrier for immobilizing the biofilm formation-inhibiting microorganisms, the natural polymer of sodium alginate (Sigma Co.) was used. Alginate is a typical material used to entrap microorganisms. A preliminary test was conducted in order to find out the alginate concentration that allows maintenance of physical strength in a membrane bioreactor for a long period of time. The concentration of alginate solution was adjusted to 4 wt % at the time of final injecting.

Rhodococcus qingshengii BH4 was proliferated by culturing for 24 hours in a shaking incubator. 200 mL of the culture was centrifuged and the supernatant was discarded thereby removing the culture medium. The remaining agglomerates of Rhodococcus qingshengii were washed with Tris-HCl 50 mM buffer (pH 7.0) and resuspended in ultrapure water. Subsequently, as shown in FIG. 11, the resuspended solution of the biofilm formation-inhibiting microorganisms was mixed with the alginate solution and injected to calcium chloride (CaCl₂) solution. As a result, a spherical fluidisable carrier having a network structure allowing efficient mass transfer was prepared through chemical crosslinking. The concentration of the alginate solution at the time of the final injection was 4 wt % when preparing the spherical fluidisable carrier. After crosslinking in 2 wt % calcium chloride (CaCl₂) solution for 1 hour, the prepared spherical fluidisable carrier was dried at room temperature for 20 hours in order to increase physical strength.

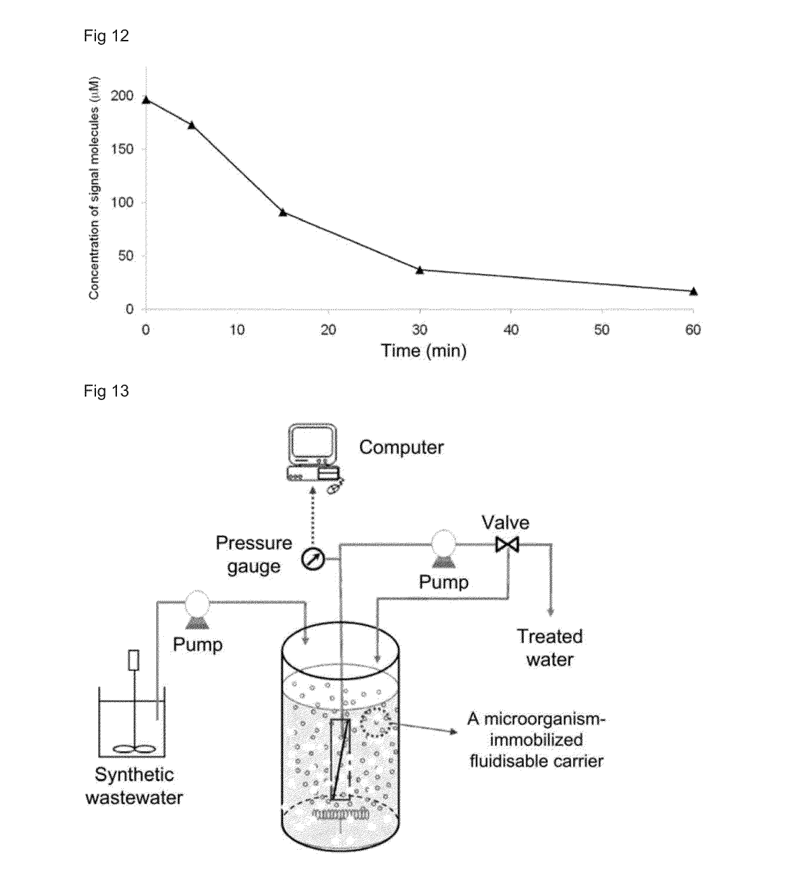

The signal molecule (AHL) decomposition activity of the spherical fluidisable carrier was measured using N-octanoyl-L-homoserine lactone (OHL) as in Embodiment 1. After adding 30 mL of Tris-HCl 50 mM buffer (pH 7) to a test tube and then injecting OHL to a concentration of 0.2 μM, the spherical fluidisable carrier was added thereto and the resulting mixture was reacted for 60 minutes in a shaking incubator of 30° C. at 200 rpm. As a result, about 92% of signal molecules were decomposed for 90 minutes by the biofilm formation-inhibiting enzyme (lactonase) produced by the biofilm formation-inhibiting microorganisms (see FIG. 12).

### EXAMPLE 1B

### Application to Membrane Bioreactor Apparatus

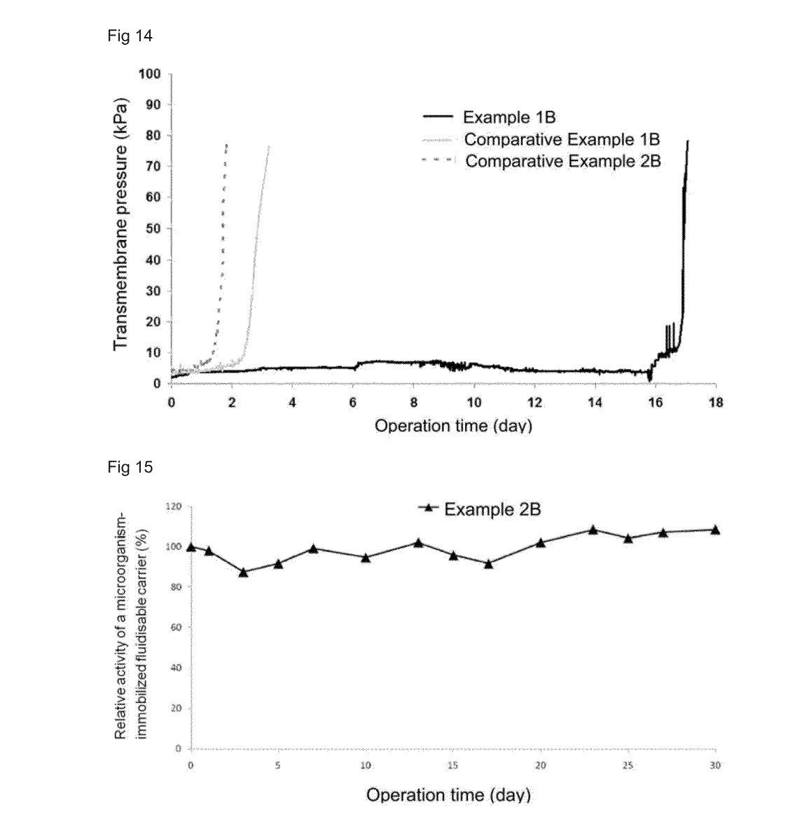

The spherical fluidisable carrier with biofilm formation-inhibiting microorganisms immobilized therein prepared in Preparation Example 1B was applied to a laboratory-scale membrane bioreactor process (see FIG. 13). Specifically, 1.6 L of activated sludge was filled in a cylindrical reactor and diffuser stone was equipped at the bottom to maintain aeration of 1 L/min. A total of 60 pieces of the spherical fluidisable carriers were placed in the reactor. For continuous operation, synthetic wastewater containing glucose as a main carbon source was introduced by an inflow pump. The chemical oxygen demand (COD) of the synthetic wastewater was about 560 ppm and hydraulic retention time was about 5.3 hours. The synthetic wastewater was filtered with a flux of about 28.7 L/m²·hr through a hollow fiber ultrafiltration membrane (Zeeweed 500, GE-Zenon, pore size 0.04 μm) submerged in the reactor. The water level of the reactor was maintained by recirculating a part of the permeate using a level controller and a 3-way-valve. The degree of membrane biofouling caused by biofilm formation on the membrane surface was represented with transmembrane pressure (TMP). The higher the transmembrane pressure, the larger is the degree of membrane biofouling. Even after operation for about 77 hours, the transmembrane pressure was no more than about 5 kPa. After operation for about 400 hours, the transmembrane pressure reached about 70 kPa (see FIG. 14).

### COMPARATIVE EXAMPLE 1B

The same procedure was repeated as Example 1B except that 60 pieces of hydrogel spherical fluidisable carriers without any microorganisms immobilized (prepared by not immobilizing the biofilm formation-inhibiting microorganisms in Preparation Example 1B) were placed in the reactor instead of the spherical fluidisable carriers with biofilm formation-inhibiting microorganisms immobilized therein. After operation for about 77 hours, the transmembrane pressure reached about 70 kPa (see FIG. 14).

### COMPARATIVE EXAMPLE 2B

The same procedure was repeated as Example 1B except that the spherical fluidisable carriers were not placed in the membrane bioreactor. After operation for about 43 hours, the transmembrane pressure reached about 70 kPa (see FIG. 14).

From Example 1B and Comparative Examples 1 B-2B, it can be seen that the membrane bioreactor apparatus in which the spherical fluidisable carriers with biofilm formation-inhibiting microorganisms immobilized therein of the present disclosure are placed (Example 1 B) exhibits remarkably decreased biofouling on the membrane surface as compared to when the spherical fluidisable carriers without the microorganisms immobilized are placed (Comparative Example 1 B) or no spherical fluidisable carrier is placed (Comparative Example 2B). This is thought of as a synergic effect of molecular biological effect of inhibiting biofilm formation by the biofilm formation-inhibiting microorganisms stably immobilized in the spherical fluidisable carrier and removal of biofilms on the membrane surface by physical washing owing to the carrier having fluidisability through submerged aeration.

### EXAMPLE 2B

### Maintenance of Activity of the Spherical Fluidisable Carrier with Biofilm Formation-Inhibiting Microorganisms Immobilized Therein

It was investigated whether the signal molecule decomposition activity of the biofilm formation-inhibiting microorganisms inside the spherical fluidisable carrier is maintained for a long period of time. Specifically, after continuous operation for 0, 1, 3, 5, 7, 10, 13, 15, 17, 20, 23, 25, 27 and 30 days in the Example 1 B, the spherical fluidisable carrier was taken out from the reactor and, followed by washing the outside of the fluidisable carrier several times with distilled water, the signal molecule decomposition activity of the biofilm formation-inhibiting microorganisms was measured according to the same procedure as Preparation Example 1 B. Relative activity was measured relative to the activity of the spherical fluidisable carrier on day 0 as 100%. Even after operation for 20days, the signal molecule decomposition activity of the spherical fluidisable carrier did not decrease but increased slightly as compared to the initial (day 0) activity (FIG. 15).

### EXAMPLE 3B

### Growth of Biofilm Formation-Inhibiting Microorganisms Inside the Spherical Fluidisable Carrier

The degree of growth of the biofilm formation-inhibiting microorganisms was investigated after the spherical fluidisable carrier was placed in a membrane bioreactor and operated for a long period of time.

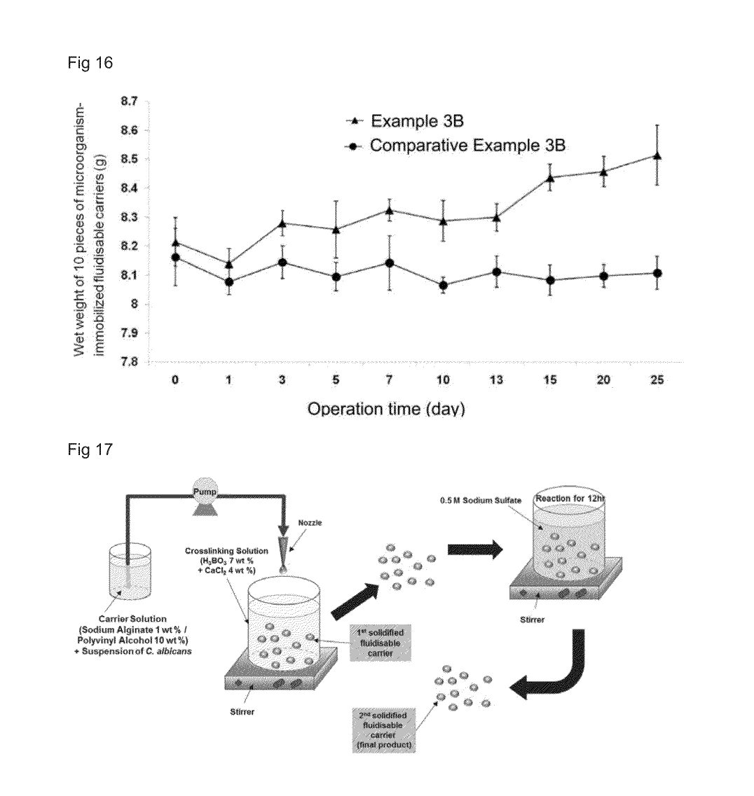

Specifically, while operating the reactor for 25 days after placing the spherical fluidisable carrier, 10 pieces of the spherical fluidisable carriers were recovered every 24 hours and, followed by washing the outside of the fluidisable carrier several times with distilled water, and wet weight was measured (Average was taken for 5 repeated measurements). 25 days later, the wet weight was increased as compared to that of the initially (day 0) entrapped biofilm formation-inhibiting microorganisms (FIG. 16).

### COMPARATIVE EXAMPLE 3B

The same procedure was repeated as Example 3B except that alginate fluidisable carrier with no biofilm formation-inhibiting microorganisms immobilized was used. There was almost no change in wet weight (FIG. 16).

From Examples 2B-3B and Comparative Example 3B, it can be seen that the biofilm formation-inhibiting microorganisms immobilized in the spherical fluidisable carrier of the present disclosure grow inside the fluidisable carrier and lead to increased wet weight. This explains why the signal molecule decomposition activity does not decrease but increase slightly.

2. Embodiment 2 Concerning a Spherical Fluidisable Carrier with Biofilm Formation-inhibiting Microorganisms (Fungi) Immobilized Therein

### PREPARATION EXAMPLE 1C

### Preparation of a Spherical Fluidisable Carrier with Biofilm Formation-Inhibiting Microorganisms (Fungi) Immobilized Therein and Measurement of Inhibition Activity of Quorum Sensing

Genetically recombined Candida albicans, one of the fungi of genus Candida, capable of secreting excessive farnesol which is a substance involved in the inhibition of AI-2 quorum sensing mechanism was used as the biofilm formation-inhibiting microorganisms.

A mixture of sodium alginate (produced by Sigma Co.) which is a typical natural polymer used for entrapping microorganisms and polyvinyl alcohol (produced by Sigma Co.) was used as a raw material of a fluidisable carrier for immobilizing the biofilm formation-inhibiting microorganisms. A resuspended solution of Candida albicans was prepared by proliferating through culturing in a shaking incubator for 24 hours, centrifugating 200 ml of the shaken culture, discarding the supernatant, removing the culture medium, and thereafter resuspending remaining agglomerates of Candida albicans in ultrapure water. As shown in FIG. 17, the resuspended solution of Candida albicans and the raw material mixture of carrier of sodium alginate/polyvinyl alcohol were mixed to prepare a carrier solution (1 wt % of sodium alginate and 10 wt % of polyvinyl alcohol), and the carrier solution was injected into an aqueous mixture solution of calcium chloride (CaCl₂, 4 wt %) and boric acid (H₃BO₃, 7 wt %) as a crosslinking solution to perform first solidification for 1 hour. Then, second solidification was performed in a 0.5 M aqueous sodium sulfate solution for 12 hours to finally prepare a spherical fluidisable carrier (average diameter: 4 mm) having a network structure allowing efficient mass transfer through internal chemical crosslinking.

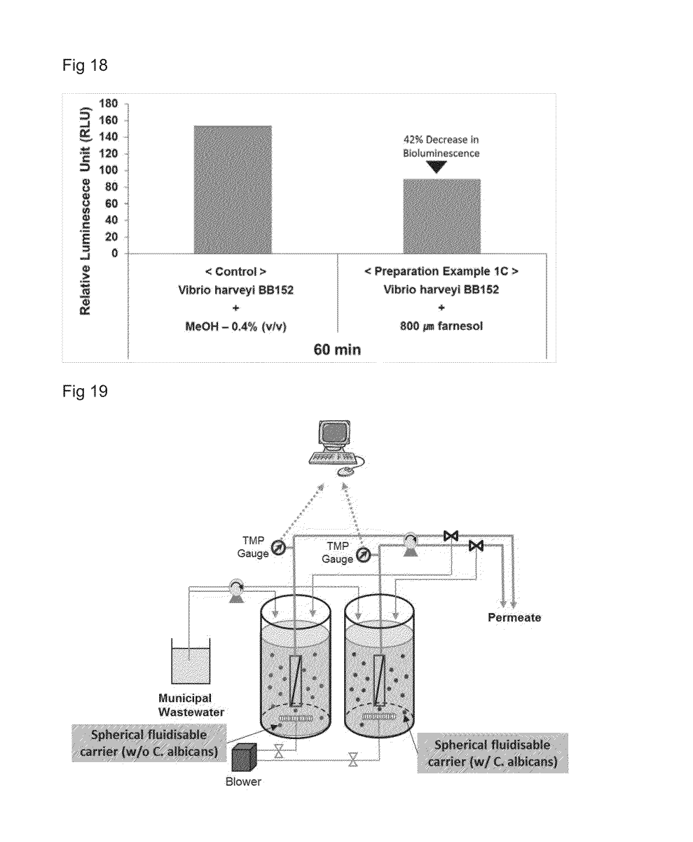

The effect of inhibition of quorum sensing for certain microorganisms (bacteria) for water treatment by farnesol secreted by the biofilm formation-inhibiting microorganisms, i.e., Candida albicans is thought to be attributed to the inhibition of quorum sensing mechanism using AI-2 signal molecules, which was indirectly assessed by means of Vibrio harveyi BB152, a bacteria producing only AI-2, and Vibrio harveyi BB170, bacteria allowing bioluminescence by specifically accepting only AI-2. Particularly, Vibrio harveyi BB152 producing only AI-2 signal molecules was inoculated in an AB medium (Autoinducer Bioassay medium, Tega et al., 2011) and cultivated to a certain degree of optical density (O.D.₆₀₀) of 0.1-0.3. Farnesol was injected to attain final concentration of 800 μM (and no farnesol was injected for its comparison), each sample was taken after reaction for 90 minutes, and the each sample was reacted with Vibrio harveyi BB170 to measure bioluminescence. As a result, the bioluminescence of Vibrio harveyi BB170 was decreased by about 42 percents for the case of not injecting the farnesol (designated as “Control”) when compared to the case of injecting the farnesol (see FIG. 18). This result was supposed to be obtained, because the farnesol inhibited the generation of AI-2 which is one kind of signal molecules for quorum sensing of certain microorganisms (bacteria for water treatment).

### EXAMPLE 1C

### Application to Membrane Bioreactor Apparatus

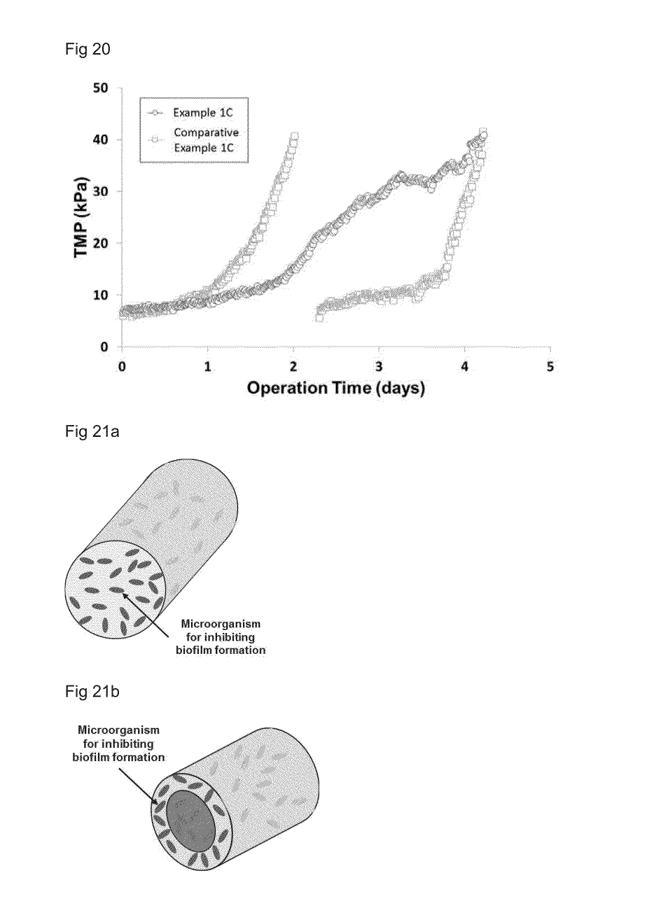

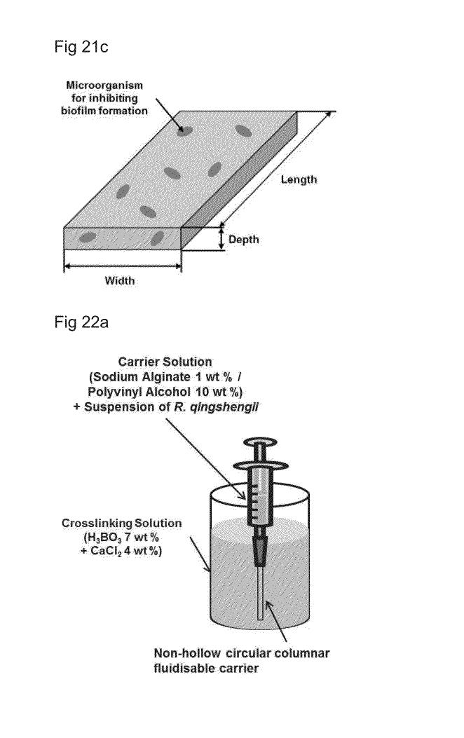

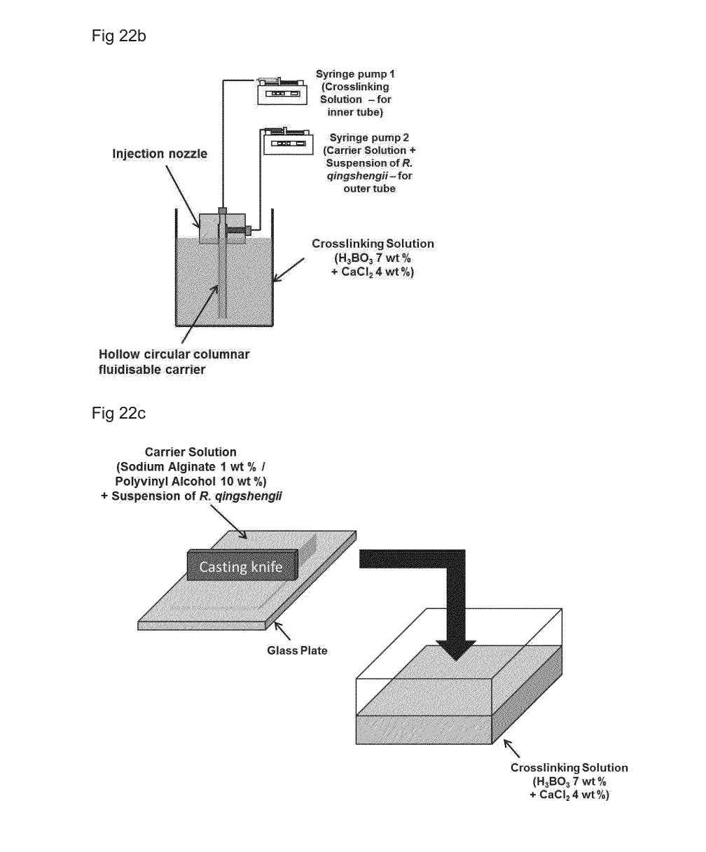

The spherical fluidisable carrier with biofilm formation-inhibiting microorganisms (Candida albicans) immobilized therein prepared in Preparation Example 1C was applied to a laboratory-scale membrane bioreactor apparatus (see FIG. 19). Particularly, 2.5 L of activated sludge was filled in a cylindrical reactor, and a diffuser stone was equipped at the bottom to maintain an aeration of 1.5 L/min. The spherical fluidisable carrier with Candida albicans immobilized therein was injected into the reactor by 0.5 v/v% of the reactor volume (corresponds to about 200-250 pieces). Municipal sewage (wastewater from the cafeteria of Seoul National University, COD: about 100-200 ppm) was injected into a reactor via an inflow pump and was operated with hydraulic retention time of about 10 hours. A submerged-type hollow fiber ultrafiltration membrane module (Zeeweed 10, GE-Zenon, pore size 0.04 μm) was installed in the reactor, and the flux of the permeate passing through the membrane was kept on about 30 L/m²·hr. The water level of the reactor was maintained by recirculating a part of the permeate using a level controller and a 3-way-valve. During the operation, a biofilm was formed on the membrane surface, and the decrease of the water permeability of the membrane due to the increase of membrane biofouling was represented by the increase of transmembrane pressure (TMP). According to the experiment results, the transmembrane pressure was merely less than about 15 kPa even after operation for 2 days and finally reached about 40 kPa after operation for 4.2 days (see FIG. 20).

### COMPARATIVE EXAMPLE 1C

The same procedure was repeated as Example 1C except for injecting a spherical fluidisable carrier with no microorganisms immobilized therein (a carrier prepared without immobilizing biofilm formation-inhibiting microorganisms therein in Preparation Example 1C) by 0.5 v/v% of the reactor volume (corresponding to about 200-250 pieces) instead of the spherical fluidisable carrier with the biofilm formation-inhibiting microorganisms (Candida albicans) immobilized therein. After operation for merely about 2 days out of 4.2 days of total operation days, the transmembrane pressure reached about 40 kPa (see FIG. 20).

That is, according to the results above of Example 1C and Comparative Example 1C, similar to the case of the spherical fluidisable carrier with biofilm formation-inhibiting microorganisms (bacteria) immobilized therein in Example 1 B, the membrane biofouling in the membrane bioreactor apparatus due to the biofilm formation on the membrane surface was remarkably relieved in the case of using the fluidisable spherical carrier with the biofilm formation-inhibiting microorganisms (fungi) immobilized therein (Example 1C) when compared to the case of the spherical fluidisable carrier with no biofilm formation-inhibiting microorganisms (fungi) immobilized therein (Comparative Example 1C). The results are thought to be obtained because the biofilm formation due to the microorganisms for water treatment on the membrane surface was restrained by the substance for inhibiting quorum sensing secreted by stably immobilized biofilm formation-inhibiting microorganisms (Candida albicans) inside the fluidisable carrier.

3. Embodiment 2 concerning a columnar fluidisable carrier with biofilm formation-inhibiting microorganisms (bacteria) immobilized therein

### PREPARATION EXAMPLE 1D

### Preparation of Non-Hollow and Hollow Circular Columnar Fluidisable Carrier with Biofilm Formation-Inhibiting Microorganisms (Bacteria) Immobilized Therein and Measurement of Decomposition Activity of Signal Molecules

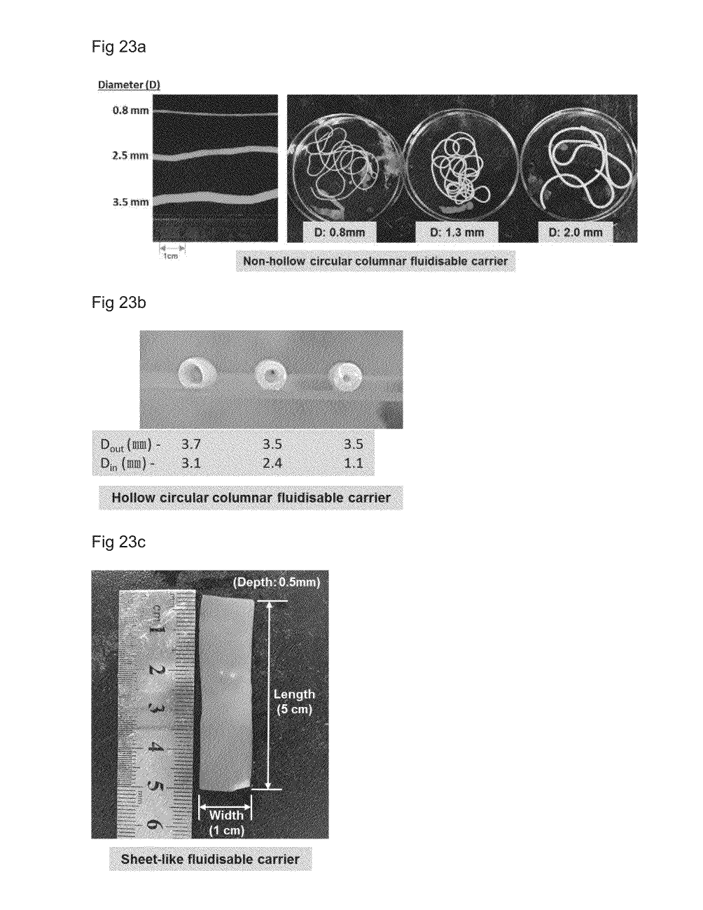

As biofilm formation-inhibiting microorganisms, Rhodococcus qingshengii BH4, isolated from sludge from the municipal wastewater treatment plant was used in the same manner described in Preparation Example 1 B. A mixture of sodium alginate (produced by Sigma Co.) which is a typical natural polymer used for entrapping microorganisms and polyvinyl alcohol (produced by Sigma Co.) was used as a raw material of a fluidisable carrier for immobilizing the biofilm formation-inhibiting microorganisms. Rhodococcus qingshengii BH₄ was proliferated by culturing for 24 hours in a shaking incubator. 200 mL of the culture was centrifuged and the supernatant was discarded thereby removing the culture medium. The remaining agglomerates of Rhodococcus qingshengii BH₄ were washed with Tris-HCl 50 mM buffer (pH 7.0) and resuspended in ultrapure water. Subsequently, as shown in FIGS. 22a and 22b, the resuspended solution of Rhodococcus qingshengii BH4 was mixed with the raw material mixture of carrier of sodium alginate/PVA to prepare a carrier solution (1 wt % of sodium alginate and 10 wt % of polyvinyl alcohol), and the carrier solution was injected to an aqueous mixture solution of calcium chloride (CaCl₂, 4 wt %) and boric acid (H₃BO₃, 7 wt %) as a crosslinking solution to perform first solidification for 1 hour. Then, second solidification was performed in 0.5 M aqueous sodium sulfate (Na₂SO₄) solution for 4 hours to finally prepare non-hollow and hollow circular columnar fluidisable carriers having a network structure allowing efficient mass transfer through internal chemical crosslinking {corresponding to “Preparation Example 1D(i)” and “Preparation Example 1 D(ii)”, respectively}. As a result, non-hollow and hollow circular columnar fluidisable carriers with various diameters/lengths were prepared as shown in FIGS. 23a and 23b.

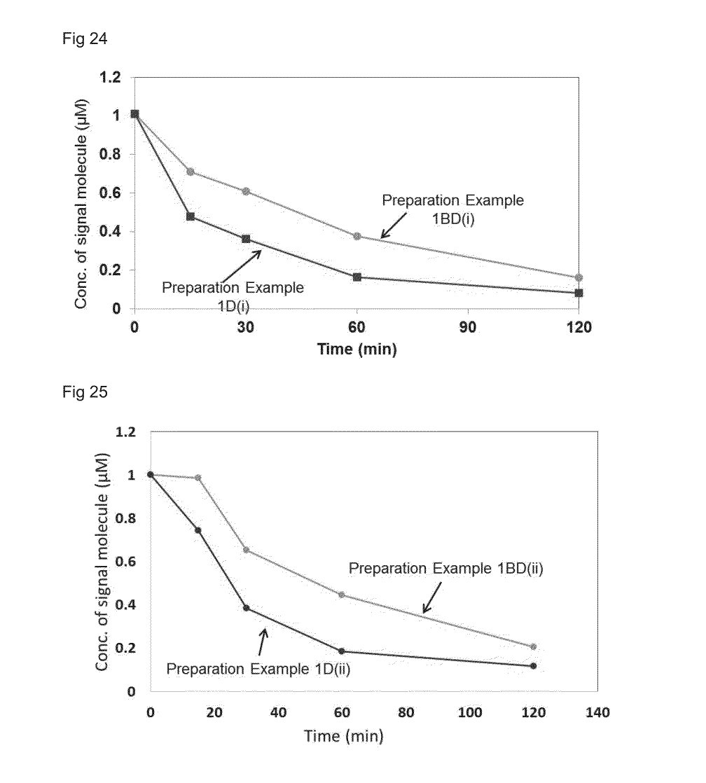

The signal molecule (AHL) decomposition activity of the columnar fluidisable carrier with biofilm formation-inhibiting microorganisms immobilized therein was measured using N-octanoyl-L-homoserine lactone (OHL) as in Preparation Example 1B. After adding 30 mL of Tris-HCl 50 mM buffer (pH 7.0) to a test tube and then injecting OHL to a concentration of 1 μM, the non-hollow and hollow columnar fluidisable carriers with biofilm formation-inhibiting microorganisms (Rhodoccocus qingshengii) immobilized therein were added thereto and the resulting mixture was reacted for 60 minutes in a shaking incubator of 30° C. at 200 rpm. The activity was measured from the amount of the signal molecule decomposed for 60 minutes by biofilm formation-inhibiting enzyme (lactonase) produced from the biofilm formation-inhibiting microorganisms (Rhodococcus qingshengii BH4). For comparison, a spherical fluidisable carrier with the same microorganisms immobilized therein was additionally prepared (hereinafter “Preparation Example 1 BD”), and the decomposition activity of the signal molecule was measured {corresponding to “Preparation Example 1 BD(i)” and “Preparation Example 1 BD(ii)”, respectively} (see FIGS. 24 and 25, respectively).

### EXAMPLE 1D(i)(a)

### Application to Membrane Bioreactor Apparatus

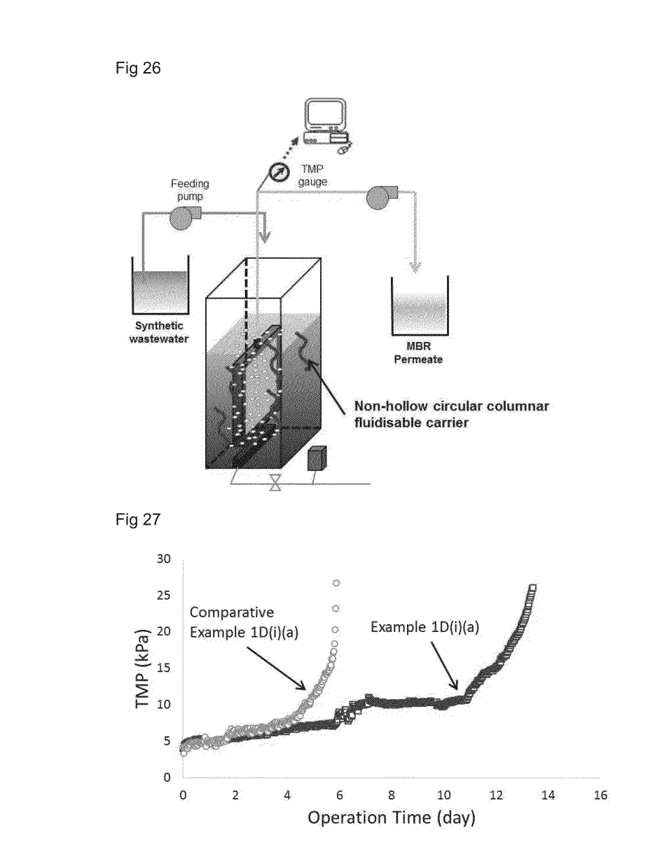

The non-hollow circular columnar fluidisable carrier with the biofilm formation-inhibiting microorganisms immobilized therein prepared in Preparation Example 1D(i) was applied to a laboratory-scale membrane bioreactor apparatus (see FIG. 26). Particularly, 4.5 L of activated sludge was filled in a rectangular reactor, and a diffuser stone was equipped at the bottom to maintain an aeration of 2 L/min. 120 pieces of non-hollow circular columnar fluidisable carriers with biofilm formation-inhibiting microorganisms immobilized therein (diameter 1-1.5 mm, length 20-23 mm, corresponding to 0.5 vol % of reactor) was injected into the reactor. For continuous operation, synthetic wastewater (COD of about 600 ppm) containing glucose as a main carbon source was introduced by an inflow pump, and operated with hydraulic retention time of about 8 hours. The synthetic wastewater was filtered with a flux of the permeate of about 37 L/m²·hr through a submerged-type flat-sheet microfiltration membrane module (C-PVC, Pure-envitech Co., pore size 0.4 μm). The water level of the reactor was maintained by recirculating a part of the permeate using a level controller and a 3-way-valve. As a result, the transmembrane pressure after operation even for about 7 days (about 168 hours) was merely about 10 kPa, and the transmembrane pressure reached about 25 kPa after operation about 14 days (about 320 hours) (see FIG. 27).

### COMPARATIVE EXAMPLE 1D(i)(a)

The same procedure was repeated as Example 1D(i)(a) except that the columnar fluidisable carrier was not injected into the membrane bioreactor. As a result, the transmembrane pressure reached about 25 kPa after operation for about 6 days (about 140 hours) (see FIG. 27).

### EXAMPLE 1D(i)(b)

### Application to Membrane Bioreactor Apparatus

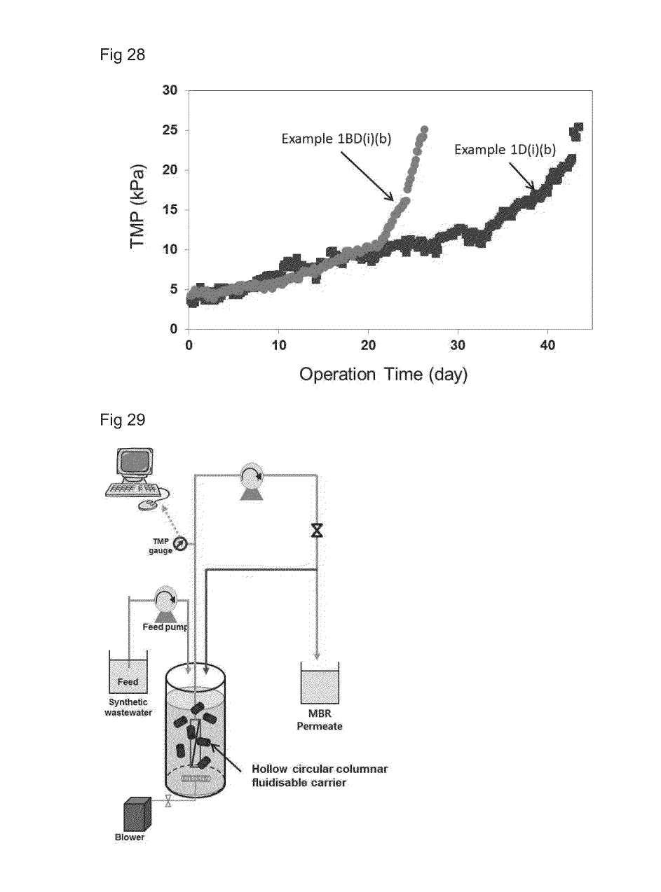

The same procedure was repeated as Example 1 D(i)(a) except that the flux of the permeate in the membrane bioreactor was kept on about 29 l/m²·hr and the dimension and the number of the columnar fluidisable carrier were changed (diameter 1-1.5 mm, length 8-12 mm, 198 pieces). As a result, the transmembrane pressure after operation even for about 23 days (about 534 hours) was merely about 10 kPa, and the transmembrane pressure reached about 25 kPa after operation about 43 days (about 1044 hours) (see FIG. 28).

For comparison, the transmembrane pressure of the same membrane module was observed by applying a spherical fluidisable carrier (average diameter: 4mm) prepared in Preparation Example 1BD to a membrane bioreactor apparatus {corresponding to “Example 1BD(i)(b)”}. More particularly, the same operation procedure was repeated as Example 1D(i)(b) except for injecting a spherical fluidisable carrier with the same biofilm formation-inhibiting microorganisms (bacteria) immobilized therein (700 pieces, 0.5 vol% relative to reactor volume) into a membrane bioreactor in Example 1 D(ii). As a result, the transmembrane pressure reached about 25 kPa after operation for about 26 days (about 630 hours) (see FIG. 28).

### EXAMPLE 1D(ii)

### Application to Membrane Bioreactor Apparatus

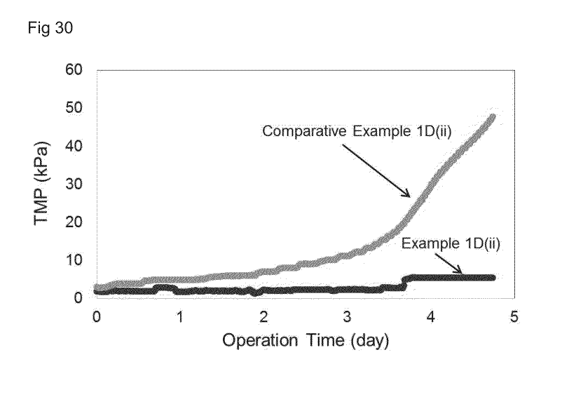

The hollow circular columnar fluidisable carrier with the biofilm formation-inhibiting microorganisms immobilized therein prepared in Preparation Example 1D(ii) was applied to a laboratory-scale membrane bioreactor apparatus (see FIG. 29). Particularly, 3 L of activated sludge was filled in a cylindrical reactor, and a diffuser stone was equipped at the bottom to maintain an aeration of 2 L/min. 280 pieces of hollow circular columnar fluidisable carriers with biofilm formation-inhibiting microorganisms immobilized therein (outer diameter 3.2 mm, inner diameter 2.0 mm, length 20 mm) was injected into a reactor. For continuous operation, synthetic wastewater containing glucose as a main carbon source (COD of about 600 ppm) was introduced by an inflow pump, and operation was performed with hydraulic retention time of about 8 hours. The synthetic wastewater was filtered with a flux of the permeate of about 21 L/m²·hr through a submerged-type hollow fiber ultrafiltration membrane module (Zeeweed 500, GE-Zenon Co., pore size 0.04 μm) installed in the reactor. The water level of the reactor was maintained by recirculating a part of the permeate using a level controller and a 3-way-valve. As a result, the transmembrane pressure after operation even for about 5 days was maintained less than or equal to about 6 kPa (see FIG. 30).

### COMPARATIVE EXAMPLE 1D(ii)

The same procedure was repeated as Example 1D(ii) except for not injecting the fluidisable carrier with the biofilm formation-inhibiting microorganisms immobilized therein into the membrane bioreactor. As a result, the transmembrane pressure after operation for about 5 days reached about 47 kPa (see FIG. 30).

That is, according to the results above of Examples 1D(i)-(ii) and Comparative Examples 1D(i)-(ii), the membrane biofouling due to the biofilm formation on the membrane surface was remarkably relieved in the case of using the columnar fluidisable carrier with the biofilm formation-inhibiting microorganisms immobilized therein was injected (Examples 1D(i) and 1D(ii)) when compared to the cases of not injecting the columnar fluidisable carrier (Comparative Examples 1D(i)(a) and 1D(ii)). The results are thought to be obtained because the biofilm formation-inhibiting microorganisms were stably immobilized in the columnar fluidisable carrier with high surface area per carrier volume, mass transfer across the carrier surface was more efficiently enhanced, and thereby molecular biological effects of efficient inhibition of the biofilm formation mechanism were attained. In addition, the effect of relieving biofouling due to physical washing of the membrane surface by movement of the columnar fluidisable carrier through submerged aeration was also supposed to be attained.

In addition, according to the results above of Examples 1D(i)(b) and 1BD(i)(b), the water permeability of the membrane was further improved for the case of using the membrane bioreactor apparatus in which the columnar fluidisable carrier of the present disclosure was injected (Example 1D(i)(b)) when compared to the spherical fluidisable carrier of the present disclosure (Example 1BD(i)(b)). It is thought to be synergistically obtained, because substantial area (surface area per carrier volume) for contact with water to be treated for the columnar fluidisable carrier was remarkably higher than that for the spherical fluidisable carrier, thereby remarkably increasing the inhibiting efficiency of the biofilm formation by more readily contacting with bacteria responsible for the biofilm formation, and the columnar fluidisable carrier has fluidisability through submerged aeration and larger substantial area for contact with the membrane surface (surface area per carrier volume), thereby expediting additional decrease of biofouling due to the detachment of the biofilm (through physical washing) on the membrane surface.

### INDUSTRIAL APPLICABILITY

When applied to an actual membrane water treatment process, the container with biofilm formation-inhibiting microorganisms immobilized therein of the present disclosure can inhibit the formation of biofilms on the membrane surface molecular biologically and, optionally, can provide an effect of physically removing membrane biofouling. As a result, decrease of permeability can be prevented, membrane cleaning cycle is lengthened, consumption of cleansers can be reduced, and lifespan of the membrane can be increased. Particularly, in the columnar or sheet-like permeable fluidisable carrier which has even larger surface area per carrier volume, biofilm formation-inhibiting microorganisms may be efficiently immobilized in the carrier (as a matrix). Thus, mass transfer across the carrier surface may be enhanced, and the formation of the biofilm may be effectively inhibited in view of the molecular biological perspective. The columnar or sheet-like permeable fluidisable carrier is not readily to be trapped even when inserted in a certain type of membrane module, and may secure a sufficient area for contact with membrane surface, thereby expediting the removal of the biofilm by efficient physical blow onto the membrane surface. Therefore, the columnar or sheet-like permeable fluidisable carrier may maximize the inhibiting/removing effect of the biofouling of the membrane surface.

And, when compared with the conventional magnetic carrier with biofilm formation-inhibiting enzyme immobilized thereon, the present disclosure is economically superior since the procedure of extracting and immobilizing enzymes is unnecessary and the apparatus for recovering the magnetic carrier is also unnecessary.

What is claimed is:

1. A container with biofilm formation-inhibiting microorganisms immobilized therein, comprising a columnar or sheet-like permeable carrier having fluidisability through submerged aeration and biofilm formation-inhibiting microorganisms immobilized in the carrier.

2. The container with biofilm formation-inhibiting microorganisms immobilized therein according to claim 1, wherein the permeable carrier is a circular columnar carrier.

3. The container with biofilm formation-inhibiting microorganisms immobilized therein according to claim 1, wherein the permeable carrier is a hollow columnar carrier.

4. The container with biofilm formation-inhibiting microorganisms immobilized therein according to claim 1, wherein the columnar permeable carrier has an aspect ratio of around 5-500, the aspect ratio being a ratio of a maximum diameter of a cross-section to length thereof.

5. The container with biofilm formation-inhibiting microorganisms immobilized therein according to claim 1, wherein the sheet-like permeable carrier has a ratio of surface area to volume (SAN) of around 5-1,000 mm⁻¹.

6. The container with biofilm formation-inhibiting microorganisms immobilized therein according to claim 1, wherein the permeable carrier comprises a hydrogel.

7. The container with biofilm formation-inhibiting microorganisms immobilized therein according to claim 1, wherein the permeable carrier comprises at least one selected from the group consisting of alginate-based, PVA-based, polyethylene glycol-based and polyurethane-based.

8. The container with biofilm formation-inhibiting microorganisms immobilized therein according to claim 1, wherein the permeable carrier has a 3-dimensional network structure through internal chemical crosslinking.

9. The container with biofilm formation-inhibiting microorganisms immobilized therein according to claim 5, wherein the permeable carrier further comprises a carbonaceous additive.

10. The container with biofilm formation-inhibiting microorganisms immobilized therein according to claim 1, wherein the permeable carrier further comprises a bio-inspired adhesive polymer additive.

11. The container with biofilm formation-inhibiting microorganisms immobilized therein according to claim 1, wherein the biofilm formation-inhibiting microorganisms are genetically recombined microorganisms or natural microorganisms.

12. The container with biofilm formation-inhibiting microorganisms immobilized therein according to claim 1, wherein the biofilm formation-inhibiting microorganisms are capable of producing enzymes for inhibiting quorum sensing.

13. The container with biofilm formation-inhibiting microorganisms immobilized therein according to claim 12, wherein the enzyme for inhibiting quorum sensing is lactonase or acylase.

14. The container with biofilm formation-inhibiting microorganisms immobilized therein according to claim 1, wherein the biofilm formation-inhibiting microorganisms are capable of producing substance for inhibiting quorum sensing.

15. The container with biofilm formation-inhibiting microorganisms immobilized therein according to claim 14, wherein the substance for inhibiting quorum sensing is farnesol.

16. A membrane water treatment apparatus, comprising: a reactor accommodating water to be treated; a membrane module for water treatment; and the container with biofilm formation-inhibiting microorganisms immobilized therein according to claim 1 in the reactor.
